(19) United States
(12) Patent Application Publication
(10) Pub. No.: US 2025/0041835 A1
(43) Pub. Date: Feb. 6, 2025

(54) A CATALYST FOR THE CONVERSION OF CO2-RICH SYNGAS TO METHANOL AND CONVENTIONAL SYNGAS TO DIMETHYL ETHER

(71) Applicant: BASF SE, Ludwigshafen am Rhein (DE)

(72) Inventors: Elias Christopher FREI, Ludwigshafen am Rhein (DE); Manuel Frederik GENTZEN, Ludwigshafen am Rhein (DE); Nils BOTTKE, Ludwigshafen am Rhein (DE); Sabine BORCHERS, Ludwigshafen am Rhein (DE); Andreas SPIEGEL, Ludwigshafen am Rhein (DE); Virginie LANVER, Ludwigshafen am Rhein (DE); Chiara BOSCAGLI, Hamburg (DE); Jan Philipp HERRMANN, Ludwigshafen am Rhein (DE)

(21) Appl. No.: 18/709,050

(22) PCT Filed: Nov. 15, 2022
(86) PCT No.: PCT/EP2022/081980

§ 371 Date: May 10, 2024

(30) Foreign Application Priority Data
Nov. 16, 2021 (EP) 21208578.1

(51) Int. Cl. B01J 23/80; C07C 29/154; C07C 31/04; C07C 41/09; C07C 43/04

(52) U.S. Cl. CPC B01J 23/80; C07C 29/154; C07C 31/04; C07C 41/09; C07C 43/043; B01J 2523/17; B01J 2523/27; B01J 2523/31; B01J 2523/41; B01J 2523/48

(57) ABSTRACT

The present invention relates to a supported copper catalyst comprising Cu, Zn, Al, Zr, Si, and O, wherein the catalyst comprises elemental copper, and wherein the catalyst displays a Zn:Si atomic ratio in the range of from 5:1 to 27:1, as well as to a precursor of the catalyst. Furthermore, the invention relates to a process for the preparation of a catalyst and catalyst precursor, respectively, as well as to a method for the conversion of CO₂-containing syngas to methanol, and to a method for the conversion of syngas to dimethyl ether which respectively employ the inventive catalyst.

### TECHNICAL FIELD

The present invention relates to a process for the preparation of a supported Cu catalyst, a supported Cu catalyst and use thereof. Further, the present invention relates to a process for the preparation of a catalyst precursor and a catalyst precursor. Yet further, the present invention relates to a method for the conversion of CO₂-containing syngas to methanol using the supported catalyst and a method for the conversion of syngas to dimethyl ether using the supported catalyst.

### INTRODUCTION

Methanol and dimethyl ether (DME) are important compounds with widespread applications for chemicals production and in the energy sector, e.g. as a substitute for gasoline or diesel fuels. The standard method of producing methanol is based on a conversion of conventional syngas with high CO and low CO₂ quantities, such that the main reaction is the conversion of CO with hydrogen to methanol according to equation (1):

CO+2H₂ [symbol] CH₃OH (1).

In such typical conversion reactions, the partial pressure of CO₂ and of H₂O as by-product of CO₂ conversion is low. Said scenario typically requires non-demanding conditions which enabling a long catalyst lifetime. The conversion of a CO₂-rich syngas is also possible wherein CO₂ is reacted with hydrogen to methanol according to equation (2):

CO₂+3H₂ [symbol] CH₃OH+H₂O (2).

If a CO₂-rich syngas is used CO₂ and H₂ exhibit a comparatively high partial pressure. Applying conversion of a CO₂-rich syngas is particularly challenging due to the reverse water-gas shift reaction, wherein H₂O is generated according to equation (3):

CO₂+H₂ [symbol] CO+H₂O (3).

Thus, a CO₂-rich syngas feed stream requires demanding conditions. Additionally, in a further reaction, which may take place in the presence of an acidic co-catalyst, generated methanol reacts to DME in a one-step synthesis. Suitable acidic co-catalysts are disclosed in EP 3727681 A1. Said reaction further increases the H₂O concentration in the reactor, upon the dehydration reaction of two CH₃OH molecules to DME according to equation 4:

2CH₃OH [symbol] CH₃OCH₃+H₂O (4)

Thus, special requirements have to be met for carrying out the conversion of CO₂-rich syngas or of a CO₂-only feed stream. Especially the catalysts used for said conversion have to fulfill certain requirements including catalyst stability. Further, these catalysts should also be suitable for the reverse water-gas shift reaction (see equation (3) above).

Typically, Cu-based catalysts are used, wherein Cu can be supported for example on ZnO and Al₂O₃. Generally, Cu-based catalysts are prepared by co-precipitation from a metal salt solution with a base solution under control of pH-value, temperature and stirring. In most of the cases a hydroxy-carbonate precursor of the used metals is obtained. The subsequent calcination then results in a defined arrangement of respective metal oxides. Usually, a catalyst for methanol preparation is pelletized. Activation takes place then prior to use in the reactor. During the activation, elemental Cu is usually generated from CuO, whereby other metal oxides stay mainly in their oxidic states.

Wu et al. disclose a study on the optimization of preparation conditions and improvement of stability of Cu/ZnO-based multicomponent catalysts for methanol synthesis from CO₂ and H₂. A Cu/ZnO-based multicomponent catalyst is disclosed therein which can be prepared by a coprecipitation method. It is particularly disclosed therein that the addition of a SiO₂ source can lead to an increase of the stability of a Cu/ZnO-based catalyst. The catalyst can be used for methanol synthesis from CO₂.

Based on the results of Wu et al. several catalysts were developed. U.S. Pat. No. 6,048,820 relates to a Cu-based catalyst and method for production thereof. The catalyst essentially comprises CuO, ZnO, Al₂O₃, and SiO₂. EP 2 857 095 A1 discloses a catalyst for methanol production, method for producing the same, and method for producing methanol, wherein the catalyst comprises Cu, Zn, Al, and Si in specific molar ratios.

In WO 2020/212681 A1 a catalyst containing CuO, ZnO, Al₂O₃ and SiO₂ is disclosed, wherein the catalyst has a BET specific surface area of greater than 105 m²/g and a Cu specific surface area of greater than 37 m²/g. The catalysts were tested with respect to their activity and stability. In Example 8 of WO 2020/212681 A1 a catalyst is disclosed representing the current state-of-the-art. Said catalyst was tested under rather standard syngas conditions (6 vol.-% CO, 6 vol.-% CO₂, 9 vol.-% N₂, and 79 vol.-% H₂) and under a low pressure (50 barg).

Thus, there was a need for an improved catalyst which is suitable in particular for the conversion of a CO₂-rich syngas or even for a CO₂-only feed stream, especially for a CO₂-rich syngas comprising greater than 10 volume-% CO₂ and having a molar ratio of CO₂ to CO of greater than 2. Especially, there was a need for a catalyst fulfilling demanding stability criteria. Further, there was a need for a novel process for the preparation of such an improved catalyst.

### DETAILED DESCRIPTION

Therefore, it was an object of the present invention to provide a novel process for the preparation of a supported Cu catalyst as well as to provide an improved supported Cu catalyst. In particular, it was an object of the present invention to provide a highly stable supported Cu catalyst for the hydrogenation of CO₂ and CO₂-enriched syngas. Especially, it was an object to provide a supported Cu catalyst showing an improved stability under certain demanding conditions being a temperature in the range of from 200 to 350° C. and a pressure in the range of from 1 to 100 bara, preferably in the range of from 40 to 85 bara, in particular for the synthesis of methanol from CO₂ according to equation (2) above and for the synthesis of DME from methanol according to equation (4) above, or a pressure below 20 bara for the reverse water-gas shift reaction according to equation 3 above.

Thus, it has surprisingly been found that an improved supported Cu catalyst can be provided, the catalyst comprising Cu, Zn, Al, Zr, Si, and O, wherein the catalyst comprises elemental copper, and wherein the catalyst displays a specific Zn:Si atomic ratio. Further, it was surprisingly found that moldings prepared from the inventive supported Cu catalyst and having a specific tristar cross-section show a comparatively lower pressure drop than conventional tablets. Further, it has surprisingly been found that such a catalyst can be prepared by a novel process wherein in particular the pH of the reaction mixture is controlled to be in a specific range.

Therefore, the present invention relates to a process for the preparation of a catalyst precursor comprising Cu, Zn, Al, Zr, Si, and O, preferably of a catalyst precursor according to any of the particular and preferred embodiments of the present invention, the process comprising:
(i) preparing a first aqueous solution S1 comprising one or more copper containing compounds, one or more zinc containing compounds, one or more aluminum containing compounds, and one or more zirconium containing compounds, wherein the pH of S1 is in the range of from 0 to 5, preferably of from 0 to 4, more preferably of from 0.5 to 3.5, more preferably of from 0.5 to 3, more preferably of from 1 to 2.5, and more preferably of from 1 to 2;
(ii) preparing a second aqueous solution S2, wherein the pH of S2 is adjusted to a value in the range of from 8.5 to 12, preferably of from 8.5 to 11, more preferably of from 8.5 to 10.5, more preferably of from 8.5 to 10, and more preferably of from 8.5 to 9.5;
(iii) preparing an aqueous mixture M comprising a solid suspended in water, comprising feeding S1 into S2 under agitation, wherein during said feeding, the pH of the aqueous mixture M resulting from said feeding is kept in the range of from 8 to 10, preferably of from 8.5 to 9.5, wherein more preferably the pH is kept at a pH of 9±0.2;
(iv) aging the aqueous mixture M obtained from (iii) for a duration in the range of from 1 to 12 h, preferably of from 1 to 6 h, more preferably of from 1.5 to 3 h, and more preferably of from 2 to 2.5 h; and
(v) separating the solid from the aqueous mixture obtained from (iv);
wherein one or more silicon containing compounds are added for preparing S2 according to (ii), or wherein one or more silicon containing compounds are added to M during the aging of M according to (iv), or wherein one or more silicon containing compounds are added for preparing S2 according to (ii) and one or more silicon containing compounds are added to the mixture M during aging of M according to (iv).

It is preferred that the one or more copper containing compounds in (i) are one or more copper salts, preferably one or more Cu(II) salts, wherein the anion of the one or more copper salts is preferably selected from the group consisting of halides, carbonate, hydrogencarbonate, sulfate, hydrogensulfate, hydroxide, nitrate, phosphate, hydogenphosphate, dihydrogenphosphate, acetate, and combinations of two or more thereof,
more preferably from the group consisting of chloride, bromide, fluoride, hydrogencarbonate, hydrogensulfate, nitrate, dihydrogenphosphate, acetate, and combinations of two or more thereof,
more preferably from the group consisting of chloride, fluoride, nitrate, acetate, and combinations of two or more thereof,
wherein more preferably the anion of the one or more copper salts is chloride and/or nitrate, preferably nitrate,
and wherein more preferably the one or more copper containing compounds comprise copper(II) nitrate, wherein more preferably the one or more copper containing compounds is copper(II) nitrate.

It is preferred that the one or more zinc containing compounds in (i) are one or more zinc salts, preferably one or more Zn(II) salts, wherein the anion of the one or more zinc salts is preferably selected from the group consisting of halides, carbonate, hydrogencarbonate, sulfate, hydrogensulfate, hydroxide, nitrate, phosphate, hydogenphosphate, dihydrogenphosphate, acetate, and combinations of two or more thereof,
more preferably from the group consisting of chloride, bromide, fluoride, hydrogencarbonate, hydrogensulfate, nitrate, dihydrogenphosphate, acetate, and combinations of two or more thereof,
more preferably from the group consisting of chloride, fluoride, nitrate, acetate, and combinations of two or more thereof,
wherein more preferably the anion of the one or more zinc salts is chloride and/or nitrate, preferably nitrate,
and wherein more preferably the one or more zinc containing compounds comprise zinc(II) nitrate, wherein more preferably the one or more zinc containing compounds is zinc(II) nitrate.

It is preferred that the one or more aluminum containing compounds in (i) are one or more aluminum salts, wherein the anion of the one or more aluminum salts is preferably selected from the group consisting of halides, sulfate, hydroxide, nitrate, and combinations of two or more thereof,
more preferably from the group consisting of chloride, fluoride, sulfate, hydroxide, nitrate, and combinations of two or more thereof,
wherein more preferably the anion of the one or more aluminum salts is chloride and/or nitrate, preferably nitrate,
and wherein more preferably the one or more aluminum containing compounds comprise aluminum nitrate, wherein more preferably the one or more aluminum containing compounds is aluminum nitrate.

It is preferred that the one or more zirconium containing compounds in (i) are one or more zirconium and/or zirconyl salts, preferably one or more Zr(IV) salts, wherein the anion of the one or more zirconium and/or zirconyl salts is preferably selected from the group consisting of halides, carbonate, hydrogencarbonate, sulfate, hydrogensulfate, hydroxide, nitrate, phosphate, hydogenphosphate, dihydrogenphosphate, acetate, and combinations of two or more thereof,
more preferably from the group consisting of chloride, bromide, fluoride, hydrogencarbonate, hydrogensulfate, nitrate, dihydrogenphosphate, acetate, and combinations of two or more thereof,
more preferably from the group consisting of chloride, fluoride, nitrate, acetate, and combinations of two or more thereof,
wherein more preferably the anion of the one or more zirconium and/or zirconyl salts is chloride and/or nitrate, preferably nitrate,
and wherein more preferably the one or more zirconium containing compounds comprise zirconium(IV) nitrate and/or zirconyl nitrate, wherein more preferably the one or more zinc containing compounds is zirconium(IV) nitrate and/or zirconyl nitrate, preferably zirconium(IV) nitrate.

It is preferred that independently from one another, the one or more silicon containing compounds in (ii) and (iv) are selected from the group consisting of silicates, preferably from the group consisting of silicate salts, more preferably from the group consisting of alkali metal silicates and mixtures thereof, wherein the alkali metal is preferably selected from the group consisting of Li, Na, K, Rb, Cs, and mixtures of two or more thereof, more preferably from the group consisting of Li, Na, K, and mixtures of two or more thereof, wherein more preferably the alkali metal is Na and/or K, preferably Na,
and wherein more preferably the one or more silicon containing compounds comprise sodium silicate, preferably sodium water glass, and more preferably Na₂SiO₃, wherein more preferably the one or more silicon containing compounds is sodium silicate, preferably sodium water glass, and more preferably Na₂SiO₃.

It is preferred that in (i), Cu, Zn, Al, and Zr are respectively contained in S1 as salts, preferably as nitrate salts.

It is preferred that the solution S1 obtained from (i) displays a Cu:Zn:Al:Zr molar ratio in the ranges of (40-65):(10-25):(15-40):(0.2-10), preferably of (45-62):(13-23):(18-35):(0.5.-5), more preferably of (50-59):(15-21):(22-30):(1-3), and more preferably of (55-56):(17-19):(25-26):(1.4-1.6).

It is preferred that in (iii) the pH of the aqueous mixture M is kept in the pH range by metering a third aqueous solution S3 into the aqueous mixture M, wherein the pH of S3 is equal to or higher than the pH of S2, wherein the pH of S3 is preferably in the range of from 11 to 14, more preferably of from 12 to 14, and more preferably of from 13 to 14.

In case where in (iii) the pH of the aqueous mixture M is kept in the pH range by metering a third aqueous solution S3 into the aqueous mixture M, wherein the pH of S3 is equal to or higher than the pH of S2, it is preferred that S3 comprises one or more bases selected from the group consisting of Bronstedt and Lewis bases, wherein preferably the one or more bases are selected from the group consisting of inorganic and organic bases, more preferably from the group of inorganic bases, wherein preferably the one or more bases are selected from the group consisting of hydroxides, carbonates, aluminates, and mixtures of two or more thereof,
more preferably from the group consisting of alkali metal hydroxides, alkali metal carbonates, alkali metal aluminates, and mixtures of two or more thereof,
more preferably from the group consisting of alkali metal carbonates, alkali metal aluminates, and mixtures of two or more thereof,
wherein the alkali metal is preferably selected from the group consisting of Li, Na, K, Rb, Cs, and mixtures of two or more thereof,
more preferably from the group consisting of Li, Na, K, and mixtures of two or more thereof,
wherein more preferably the alkali metal is Na and/or K, preferably Na,
and wherein more preferably the one or more bases comprise sodium carbonate and/or sodium hydroxide, preferably sodium carbonate and sodium hydroxide, wherein more preferably the one or more bases are sodium carbonate and/or sodium hydroxide, preferably sodium carbonate and sodium hydroxide.

It is preferred that adding in (iii) is conducted continuously or intermittently, preferably continuously.

It is preferred that for keeping the pH of the aqueous mixture M in the pH range in (iii), the pH of the aqueous mixture M is semi-continuously or continuously monitored, preferably continuously monitored.

It is preferred that agitation in (iii) is achieved by stirring.

It is preferred that aging in (iv) is conducted at a temperature in the range of from 5 to 75° C., preferably of from 15 to 70° C., more preferably of from 25 to 65° C., more preferably of from 40 to 60° C., and more preferably of from 45 to 55° C.

It is preferred that in (iv) the sodium water glass is added after a period of aging in the range of from 0.5-11.5 h, wherein preferably the sodium water glass is optionally added after 1 h of aging.

It is preferred that the aqueous mixture M obtained from (iii) or the aged aqueous mixture obtained from (iv) displays a Cu:Zn:Al:Zr:Si molar ratio in the ranges of (40-65):(10-25):(15-40):(0.2-10):(0.1-5), preferably of (45-62):(13-23):(18-35):(0.5.-5):(0.3-4.5), more preferably of (50-59):(15-21):(22-30):(1-3):(0.8-4), and more preferably of (55-56):(17-19):(25-26):(1.4-1.6):(1.2-3.6).

It is preferred that separation in (v) is achieved by filtration.

It is preferred that the process further comprises:
(vi) washing the solid obtained from (v); and/or, preferably and
(vii) drying the solid obtained from (v) or (vi), preferably at a temperature in the range of from 80 to 150° C., preferably of from 90 to 140° C., more preferably of from 105 to 130° C., and more preferably of from 115 to 125° C.

In case where the process further comprises (vi), it is preferred that washing in (vi) is performed with deionized water.

In case where the process further comprises (vi) and/or (vii), it is preferred that drying in (vii) is conducted for a duration in the range of from 1 to 48 h, preferably of from 6 to 36 h, more preferably of from 12 to 30 h, and more preferably of from 18 to 24 h.

It is preferred that the process further comprises:
(ix) calcining the solid obtained from (v), (vi), or (vii), preferably at a temperature in the range of from 400 to 750° C., preferably of from 500 to 700° C., and more preferably of from 550 to 650° C.

In case where the process further comprises (ix), it is preferred that calcining in (ix) is conducted for a duration in the range of from 0.5 to 12 h, preferably from 1 to 6 h, and more preferably from 1.5 to 2.5 h.

In case where the process further comprises (ix), it is preferred that calcining in (ix) is performed in an atmosphere containing oxygen, preferably in an atmosphere containing air, wherein more preferably calcining in (ix) is performed in air.

The present invention also relates to a catalyst precursor comprising Cu, Zn, Al, Zr, Si, and O, preferably according to any of the particular and preferred embodiments of the present invention, as obtainable or obtained according to the process of any of the particular and preferred embodiments of the present invention.

The present invention also relates to a process for the preparation of a supported copper catalyst comprising Cu, Zn, Al, Zr, Si, and O, preferably of a supported copper catalyst according to any of the particular and preferred embodiments of the present invention, wherein the catalyst comprises elemental copper, the process comprising:
(1) preparing a catalyst precursor comprising Cu, Zn, Al, Zr, Si, and O, according to the process of any of the particular and preferred embodiments of the present invention for the preparation of a catalyst precursor including a calcination step (ix);
(2) reducing the catalyst precursor obtained from (1) in an atmosphere comprising hydrogen for obtaining the supported copper catalyst.

It is preferred that reduction in (2) is conducted at a temperature in the range of from 150 to 350° C. preferably of from 170 to 300° C., and more preferably of from 170 to 230° C.

It is preferred that the atmosphere in (2) comprises from 0.25 to 80 vol.-% H₂, preferably from 0.5 to 50 vol.-% H₂, more preferably from 0.5 to 30 vol.-% H₂, more preferably from 1 to 10 vol.-% H₂, and more preferably from 2 to 5 vol.-% H₂.

It is preferred that the atmosphere in (2) comprises from 99.75 to 20 vol.-% of an inert gas, preferably from 99.5 to 50 vol.-%, more preferably from 99.5 to 70 vol.-%, more preferably from 99 to 90 vol.-%, and more preferably from 98 to 95 vol.-%.

In case where the atmosphere in (2) comprises from 99.75 to 20 vol.-% of an inert gas, it is preferred that the inert gas comprises one or more gases selected from the group consisting of noble gases, nitrogen gas, and methane, preferably from the group consisting of He, Ar, Ne, N₂, and CH4, wherein more preferably the inert gas comprises N₂, wherein more preferably the inert gas is N₂.

The present invention also relates to a supported copper catalyst comprising Cu, Zn, Al, Zr, Si, and O, preferably according to any of the particular and preferred embodiments of the present invention, as obtainable or obtained according to any of the particular and preferred embodiments of the inventive process for its preparation, wherein the catalyst comprises elemental copper.

The present invention also relates to a catalyst precursor comprising Cu, Zn, Al, Zr, Si, and O, preferably as obtainable or obtained according to any of the particular and preferred embodiments of the inventive process for its preparation, wherein the catalyst precursor displays a Zn:Si atomic ratio in the range of from 5:1 to 27:1, preferably of from 5.5:1 to 25:1, more preferably of from 6:1 to 20:1, more preferably of from 6.5:1 to 15:1, more preferably of from 7:1 to 12:1, more preferably of from 7.5:1 to 10:1, and more preferably of from 8:1 to 9:1.

It is preferred that the catalyst precursor comprises one or more hydroxycarbonate mixed oxides comprising two or more of Cu, Zn, and Al, preferably two or more of Cu, Zn, Al, and Zr, wherein more preferably the catalyst precursor comprises one or more hydroxycarbonate mixed oxides of Cu, Zn, and Al, preferably of Cu, Zn, Al, and Zr.

It is preferred that the catalyst precursor comprises CuO.

It is preferred that the catalyst precursor comprises Zr-modified Cu₃Zn₃Al₂(OH)₁₆CO₃, preferably a Zr-modified Cu₃Zn₃Al₂(OH)₁₆CO₃·4H₂O, wherein the 003 reflection in the x-ray diffractogram of the Zr-modified Cu₃Zn₃Al₂(OH)₁₆CO₃ is shifted to higher ° 2Theta values compared to the x-ray diffractogram of Cu₃Zn₃Al₂(OH)₁₆CO₃, wherein the 003 reflection in the x-ray diffractogram of the Zr-modified Cu₃Zn₃Al₂(OH)₁₆CO₃ is located in the range of from 11 to 13.5° 2Theta, wherein the x-ray diffractogram is preferably determined according to Reference Example 2.

Alternatively, it is preferred that the catalyst precursor, and preferably the catalyst precursor as obtainable or obtained according to the process of any of the particular and preferred embodiments of the present invention including a calcination step (ix), comprises one or more oxides of Cu, Zn, Al, Zr, and Si, wherein the catalyst precursor preferably comprises one or more oxides selected from the group consisting of CuO, ZnO, ZnAl₂O₄, and CuAl₂O₄, wherein more preferably the catalyst precursor comprises CuO, ZnO, and ZnAl₂O₄, or CuO, ZnO, and CuAl₂O₄, or CuO, ZnO, ZnAl₂O₄, and CuAl₂O₄. According to said preferred embodiments, it is further preferred that the catalyst precursor comprises CuO in an amount in the range of from 50 to 70 wt.-% calculated as the oxide CuO and based on the sum of the weights of the oxides of Cu, Zn, Al, Zr, and Si contained in the catalyst precursor, calculated as CuO, ZnO, Al₂O₃, ZrO₂, and SiO₂, wherein preferably, the catalyst precursor comprises CuO in an amount in the range of from 55 to 65 wt.-%, and more preferably of from 58 to 62 wt.-%. Furthermore, it is further preferred according to said preferred embodiments that the catalyst precursor comprises SiO₂.

It is preferred that the catalyst precursor displays a Cu:Zn:Al:Zr:Si molar ratio in the ranges of (40-65):(10-25):(15-40):(0.2-10):(0.1-5), preferably of (45-62):(13-23):(18-35):(0.5.-5):(0.3-4.5), more preferably of (50-59):(15-21):(22-30):(1-4):(0.8-4), and more preferably of (55-56):(17-19):(25-26):(1.4-3):(1.2-3.6).

It is preferred that from 95 to 100 wt.-% of the catalyst precursor consists of Cu, Zn, Al, Zr, Si, and O, preferably from 97 to 100 wt.-%, more preferably from 98 to 100 wt.-%, more preferably from 99 to 100 wt.-%, more preferably from 99.5 to 100 wt.-%, and more preferably from 99.9 to 100 wt.-%.

The present invention also relates to a supported copper catalyst comprising Cu, Zn, Al, Zr, Si, and O, preferably as obtainable or obtained according to any of the particular and preferred embodiments of the inventive process for its preparation, wherein the catalyst comprises elemental copper, and wherein the catalyst displays a Zn:Si atomic ratio in the range of from 5:1 to 27:1, preferably of from 5.5:1 to 25:1, more preferably of from 6:1 to 20:1, more preferably of from 6.5:1 to 15:1, more preferably of from 7:1 to 12:1, more preferably of from 7.5:1 to 10:1, and more preferably of from 8:1 to 9:1.

It is preferred that the supported copper catalyst comprises one or more oxides of Zn, Al, Zr, and Si, wherein the supported copper catalyst preferably comprises one or more oxides selected from the group consisting of ZnO, ZnAl₂O₄, and CuAl₂O₄, wherein more preferably the supported copper catalyst comprises ZnO and ZnAl₂O₄, or ZnO and CuAl₂O₄, or ZnO, ZnAl₂O₄, and CuAl₂O₄.

It is preferred that the supported copper catalyst displays a BET surface area of 130 m²/g or less, and preferably displays a BET surface area within the range of from 60 to 130 m²/g, wherein the BET surface area is preferably determined according to Reference Example 1.

It is preferred that the supported copper catalyst displays an copper surface area in the range of from 5 to 15 m²/g, preferably for from 10 to 13 m²/g, wherein the copper surface area is determined according to Reference Example 3.

It is preferred that the supported copper catalyst comprises SiO₂.

It is preferred that the supported copper catalyst displays a Cu:Zn:Al:Zr:Si molar ratio in the ranges of (40-65):(10-25):(15-40):(0.2-10):(0.1-5), preferably of (45-62):(13-23):(18-35):(0.5.-5):(0.3-4.5), more preferably of (50-59):(15-21):(22-30):(1-4):(0.8-4), and more preferably of (55-56):(17-19):(25-26):(1.4-3):(1.2-3.6).

It is preferred that from 95 to 100 wt.-% of the supported copper catalyst consists of Cu, Zn, Al, Zr, Si, and O, preferably from 97 to 100 wt.-%, more preferably from 98 to 100 wt.-%, more preferably from 99 to 100 wt.-%, more preferably from 99.5 to 100 wt.-%, and more preferably from 99.9 to 100 wt.-%.

It is preferred that the supported copper catalyst is in the form of a molding having a height H and a tristar cross-section with rounded tips and with rounded corners. Schematical drawings of such a molding are shown in FIGS. 6 and 7, wherein the height H of the molding is shown in FIG. 7.

According to the present invention, a molding is to be understood as a three-dimensional entity obtained from a shaping process; accordingly, the term “molding” is used synonymously with the term “shaped body”.

In the case where the supported copper catalyst is in the form of a molding having a height H and a tristar cross-section with rounded tips and with rounded corners, it is preferred that the tip basis b (see FIG. 6) of said tristar cross-section is in the range of from 2.00 to 2.60 mm, more preferably in the range of from 2.10 to 2.50 mm, more preferably in the range of from 2.20 to 2.40 mm, more preferably in the range of from 2.25 to 2.35 mm.

Further in the case where the supported copper catalyst is in the form of a molding having a height H and a tristar cross-section with rounded tips and with rounded corners, it is preferred that the tip height i (see FIG. 6) of said tristar cross-section is in the range of from 2.55 to 3.15 mm, more preferably in the range of from 2.65 to 3.05 mm, more preferably in the range of from 2.75 to 2.95 mm, more preferably in the range of from 2.80 to 2.90 mm.

Further in the case where the supported copper catalyst is in the form of a molding having a height H and a tristar cross-section with rounded tips and with rounded corners, it is preferred that the height h (see FIG. 6) of said tristar cross-section is in the range of from 5.40 to 6.00 mm, more preferably in the range of from 5.50 to 5.90 mm, more preferably in the range of from 5.60 to 5.80 mm, more preferably in the range of from 5.65 to 5.75 mm.

Further in the case where the supported copper catalyst is in the form of a molding having a height H and a tristar cross-section with rounded tips and with rounded corners, it is preferred that the chord length k (see FIG. 6) of said tristar cross-section is in the range of from 6.00 to 6.60 mm, more preferably in the range of from 6.10 to 6.50 mm, more preferably in the range of from 6.20 to 6.40 mm, more preferably in the range of from 6.25 to 6.35 mm.

Further in the case where the supported copper catalyst is in the form of a molding having a height H and a tristar cross-section with rounded tips and with rounded corners, it is preferred that said tristar cross-section has an inner angle α (see FIG. 6) in the range of from 5 to 19°, more preferably in the range of from 8 to 16°, more preferably in the range of from 10 to 14°, more preferably in the range of from 11 to 13°.

In the context of the present invention, the inner angle α defines the relation of two axes forming a portion of a tip of a tristar cross-section. According to the present invention each tip is rounded, in particular by an arc of a cycle having a specific radius.

Further in the case where the supported copper catalyst is in the form of a molding having a height H and a tristar cross-section with rounded tips and with rounded corners, it is preferred that each of the tips of said tristar cross-section independently from each other is rounded by an arc of a circle having a radius in the range of from 0.65 to 1.25 mm, more preferably in the range of from 0.75 to 1.15 mm, more preferably in the range of from 0.85 to 1.05 mm, more preferably in the range of from 0.90 to 1.00 mm.

Further in the case where the supported copper catalyst is in the form of a molding having a height H and a tristar cross-section with rounded tips and with rounded corners, it is preferred that the distance between the geometric center C (see FIG. 6) of said tristar cross-section and each of the tips of said tristar cross-section independently from each other is in the range of from 2.0 to 5.0 mm, more preferably in the range of from 2.7 to 4.3 mm, more preferably in the range of from 3.2 to 3.8 mm, more preferably in the range of from 3.4 to 3.6 mm.

Further in the case where the supported copper catalyst is in the form of a molding having a height H and a tristar cross-section with rounded tips and with rounded corners, it is preferred that said tristar cross-section has an outer angle β (see FIG. 6) in the range of from 125 to 155°, more preferably in the range of from 135 to 145°, more preferably in the range of from 127 to 137°, more preferably in the range of from 130 to 134°.

In the context of the present invention, the outer angle β defines the relation of an axis of one tip to an axis of an adjacent tip of a tristar cross-section, thereby forming a corner. According to the present invention each corner is rounded, in particular by an arc of a cycle having a specific radius.

Further in the case where the supported copper catalyst is in the form of a molding having a height H and a tristar cross-section with rounded tips and with rounded corners, it is preferred that each of the corners of said tristar cross-section independently from each other is rounded by an arc of a circle having a radius in the range of from 0.65 to 1.25 mm, more preferably in the range of from 0.75 to 1.15 mm, more preferably in the range of from 0.85 to 1.05 mm, more preferably in the range of from 0.90 to 1.00 mm.

Further in the case where the supported copper catalyst is in the form of a molding having a height H and a tristar cross-section with rounded tips and with rounded corners, it is preferred that the height H of said molding is in the range of from 3.0 to 11.0 mm, more preferably in the range of from 3.5 to 10.5 mm, more preferably in the range of from 4.5 to 9.5 mm, more preferably in the range of from 5.5 to 8.5 mm, more preferably in the range of from 6.5 to 7.5 mm.

The present invention also relates to a method for the conversion of CO₂-containing syngas to methanol, the method comprising:
(A) providing a supported copper catalyst according to any of the particular and preferred embodiments of the present invention;
(B) preparing a gas mixture comprising CO, H₂, and CO₂;
(C) contacting the supported copper catalyst provided in (A) with the gas mixture prepared in (B), wherein the contacting is performed at a temperature in the range of from 200 to 350° C., preferably of from 230 to 260° C.

It is preferred that contacting in (B) is performed at a pressure in the range of from 1 to 100 bara, preferably in the range of from 40 to 85 bara, more preferably from 70 to 82 bara, and
more preferably from 74 to 81 bara.

It is preferred that the gas mixture prepared in (B) comprises from 10 to 24 vol.-% CO₂, preferably from 11 to 20 vol.-%, more preferably from 12 to 19 vol.-%, and more preferably from 15 to 18 vol.-%.

It is preferred that the gas mixture prepared in (B) comprises from 0.5 to 7 vol.-% CO, preferably from 0.8 to 4 vol.-%, and more preferably from 1 to 2 vol.-%.

It is preferred that the gas mixture prepared in (B) displays a CO₂:CO molar ratio in the range of from 2 to 20, preferably for from 3 to 17, more preferably for from 5 to 15, and more preferably for from 7 to 13.

It is preferred that the gas mixture prepared in (B) comprises from 50 to 90 vol.-% H₂, preferably from 55 to 87 vol.-%, more preferably from 60 to 85 vol.-%, and more preferably from 65 to 83 vol.-%.

It is preferred that the gas mixture prepared in (B) comprises from 0.1 to 40 vol.-% of an inert gas, preferably from 0.3 to 30 vol.-%, more preferably from 0.5 to 25 vol.-%, more preferably from 0.8 to 20 vol.-%, and more preferably from 1 to 15 vol.-%.

In case where the gas mixture prepared in (B) comprises from 0.1 to 40 vol.-% of an inert gas, it is preferred that the inert gas comprises one or more gases selected from the group consisting of noble gases and nitrogen gas, preferably from the group consisting of He, Ar, Ne, CH₄, and N₂, more preferably from the group consisting of Ar, CH₄, N₂, wherein more preferably the inert gas comprises CH₄ and N₂, wherein more preferably the inert gas is CH₄ and N₂.

The present invention also relates to a method for the conversion of syngas to dimethyl ether, the method comprising:
(A) mixing a supported copper catalyst according to any of the particular and preferred embodiments of the present invention with an acidic co-catalyst;
(B) preparing a gas mixture comprising CO, H₂, and CO₂;
(C) contacting the catalyst mixture prepared in (A) with the gas mixture prepared in (B),
wherein the contacting is performed at a temperature in the range of from 200 to 300° C. preferably of from 250 to 270° C.

Within the meaning of the present invention, an acidic co-catalyst is a solid catalyst comprising acid sites. In principle, any solid catalyst comprising acid sites may be used, provided that it is suitable for catalyzing the dehydration of methanol to dimethyl ether. According to the present invention, it is preferred that the acidic co-catalyst is a catalyst comprising or consisting of methanol-to-dimethyl ether catalyst particles according to any of the particular or preferred embodiments disclosed in EP3727681A1, i.e. a catalyst comprising or consisting of methanol-to-dimethyl ether catalyst particles which comprises a catalytically active component, selected from the group consisting of
(i) acidic alumosilicate, silicate, zeolite, aluminium oxide like gamma-alumina or mixtures thereof,
(ii) acidic aluminium hydroxide, aluminium oxide hydroxide, and gamma-aluminium oxide with 0.1 to 20 weight % of niobium, tantalum, phosphorous or boron, based on the catalytically active component, or mixtures thereof or
(iii) acidic niobium oxide, tantalum oxide, titanium oxide, zirconium oxide, silicon oxide, aluminium phosphate, niobium phosphate, or mixtures thereof,
wherein the methanol-to-dimethyl ether catalyst particles furthermore comprise at least one transition metal, and any preferred or particularly preferred embodiments thereof as disclosed in the claims and the description of EP3727681A1

It is preferred that contacting in (B) is performed at a pressure in the range of from 1 to 100 bara, preferably in the range of from 50 to 85 bara, more preferably of from 60 to 75 bara.

It is preferred that the gas mixture prepared in (B) comprises 25 vol.-% or less of CO₂, preferably from 2 to 15 vol.-% CO₂, more preferably from 3 to 10 vol.-%, and more preferably from 4 to 6 vol.-%.

It is preferred that the gas mixture prepared in (B) comprises from 2 to 30 vol.-% CO, preferably from 5 to 29 vol.-%, more preferably from 10 to 28 vol.-%, more preferably from 20 to 27 vol.-%, and more preferably from 24 to 26 vol.-%.

It is preferred that the gas mixture prepared in (B) displays a CO₂:CO molar ratio of 5 or less, preferably a CO₂:CO molar ratio in the range of from 0.05 to 2, more preferably in the range of from 0.05 to 1, more preferably of from 0.1 to 0.5, and more preferably of from 0.15 to 0.25.

It is preferred that the gas mixture prepared in (B) comprises from 30 to 70 vol.-% H₂, preferably from 40 to 65 vol.-%, and more preferably from 50 to 60 vol.-%.

It is preferred that the gas mixture prepared in (B) comprises from 1 to 30 vol.-% of an inert gas, preferably from 5 to 25 vol.-%, and more preferably from 10 to 20 vol.-%.

In case where the gas mixture prepared in (B) comprises from 1 to 30 vol.-% of an inert gas, it is preferred that the inert gas comprises one or more gases selected from the group consisting of noble gases and nitrogen gas, preferably from the group consisting of He, Ar, Ne, CH₄, and N₂, wherein more preferably the inert gas comprises CH₄ and N₂, wherein more preferably the inert gas is N₂.

The present invention also relates to the use of a supported copper catalyst according to any of the particular and preferred embodiments of the present invention as a reverse water-gas shift catalyst, in the reforming of methanol, in the conversion of CO₂-containing syngas to methanol, in the reforming of dimethyl ether, and in the conversion of syngas to dimethyl ether.

The unit bara refers to an absolute pressure wherein 1 bar equals 10⁵ Pa.

The present invention is further illustrated by the following set of embodiments and combinations of embodiments resulting from the dependencies and back-references as indicated.

In particular, it is noted that in each instance where a range of embodiments is mentioned, for example in the context of a term such as “any one of embodiments (1) to (4)”, every embodiment in this range is meant to be explicitly disclosed for the skilled person, i.e. the wording of this term is to be understood by the skilled person as being synonymous to “any one of embodiments (1), (2), (3), and (4)”. Further, it is explicitly noted that the following set of embodiments is not the set of claims determining the extent of protection, but represents a suitably structured part of the description directed to general and preferred aspects of the present invention.
1. A process for the preparation of a catalyst precursor comprising Cu, Zn, Al, Zr, Si, and O, preferably of a catalyst precursor according to any of embodiments 31 to 39, the process comprising:
   (i) preparing a first aqueous solution S1 comprising one or more copper containing compounds, one or more zinc containing compounds, one or more aluminum containing compounds, and one or more zirconium containing compounds, wherein the pH of S1 is in the range of from 0 to 5, preferably of from 0 to 4, more preferably of from 0.5 to 3.5, more preferably of from 0.5 to 3, more preferably of from 1 to 2.5, and more preferably of from 1 to 2;
   (ii) preparing a second aqueous solution S2, wherein the pH of S2 is adjusted to a value in the range of from 8.5 to 12, preferably of from 8.5 to 11, more preferably of from 8.5 to 10.5, more preferably of from 8.5 to 10, and more preferably of from 8.5 to 9.5;
   (iii) preparing an aqueous mixture M comprising a solid suspended in water, comprising feeding S1 into S2 under agitation, wherein during said feeding, the pH of the aqueous mixture M resulting from said feeding is kept in the range of from 8 to 10, preferably of from 8.5 to 9.5, wherein more preferably the pH is kept at a pH of 9±0.2;
   (iv) aging the aqueous mixture M obtained from (iii) for a duration in the range of from 1 to 12 h, preferably of from 1 to 6 h, more preferably of from 1.5 to 3 h, and more preferably of from 2 to 2.5 h; and
   (v) separating the solid from the aqueous mixture obtained from (iv);
   wherein one or more silicon containing compounds are added for preparing S2 according to (ii), or wherein one or more silicon containing compounds are added to M during the aging of M according to (iv), or wherein one or more silicon containing compounds are added for preparing S2 according to (ii) and one or more silicon containing compounds are added to the mixture M during aging of M according to (iv).
2. The process of embodiment 1, wherein the one or more copper containing compounds in (i) are one or more copper salts, preferably one or more Cu(II) salts, wherein the anion of the one or more copper salts is preferably selected from the group consisting of halides, carbonate, hydrogencarbonate, sulfate, hydrogensulfate, hydroxide, nitrate, phosphate, hydogenphosphate, dihydrogenphosphate, acetate, and combinations of two or more thereof,
   more preferably from the group consisting of chloride, bromide, fluoride, hydrogencarbonate, hydrogensulfate, nitrate, dihydrogenphosphate, acetate, and combinations of two or more thereof,
   more preferably from the group consisting of chloride, fluoride, nitrate, acetate, and combinations of two or more thereof,
   wherein more preferably the anion of the one or more copper salts is chloride and/or nitrate, preferably nitrate,
   and wherein more preferably the one or more copper containing compounds comprise copper(II) nitrate, wherein more preferably the one or more copper containing compounds is copper(II) nitrate.
3. The process of embodiment 1 or 2, wherein the one or more zinc containing compounds in (i) are one or more zinc salts, preferably one or more Zn(II) salts, wherein the anion of the one or more zinc salts is preferably selected from the group consisting of halides, carbonate, hydrogencarbonate, sulfate, hydrogensulfate, hydroxide, nitrate, phosphate, hydogenphosphate, dihydrogenphosphate, acetate, and combinations of two or more thereof,
   more preferably from the group consisting of chloride, bromide, fluoride, hydrogencarbonate, hydrogensulfate, nitrate, dihydrogenphosphate, acetate, and combinations of two or more thereof,
   more preferably from the group consisting of chloride, fluoride, nitrate, acetate, and combinations of two or more thereof,
   wherein more preferably the anion of the one or more zinc salts is chloride and/or nitrate, preferably nitrate,
   and wherein more preferably the one or more zinc containing compounds comprise zinc(II) nitrate, wherein more preferably the one or more zinc containing compounds is zinc(II) nitrate.
4. The process of any of embodiments 1 to 3, wherein the one or more aluminum containing compounds in (i) are one or more aluminum salts, wherein the anion of the one or more aluminum salts is preferably selected from the group consisting of halides, sulfate, hydroxide, nitrate, and combinations of two or more thereof,
   more preferably from the group consisting of chloride, fluoride, sulfate, hydroxide, nitrate, and combinations of two or more thereof,
   wherein more preferably the anion of the one or more aluminum salts is chloride and/or nitrate, preferably nitrate,
   and wherein more preferably the one or more aluminum containing compounds comprise aluminum nitrate, wherein more preferably the one or more aluminum containing compounds is aluminum nitrate.
5. The process of any of embodiments 1 to 4, wherein the one or more zirconium containing compounds in (i) are one or more zirconium and/or zirconyl salts, preferably one or more Zr(IV) salts, wherein the anion of the one or more zirconium and/or zirconyl salts is preferably selected from the group consisting of halides, carbonate, hydrogencarbonate, sulfate, hydrogensulfate, hydroxide, nitrate, phosphate, hydogenphosphate, dihydrogenphosphate, acetate, and combinations of two or more thereof,
   more preferably from the group consisting of chloride, bromide, fluoride, hydrogencarbonate, hydrogensulfate, nitrate, dihydrogenphosphate, acetate, and combinations of two or more thereof,
   more preferably from the group consisting of chloride, fluoride, nitrate, acetate, and combinations of two or more thereof,
   wherein more preferably the anion of the one or more zirconium and/or zirconyl salts is chloride and/or nitrate, preferably nitrate,
   and wherein more preferably the one or more zirconium containing compounds comprise zirconium(IV) nitrate and/or zirconyl nitrate, wherein more preferably the one or more zinc containing compounds is zirconium(IV) nitrate and/or zirconyl nitrate, preferably zirconium(IV) nitrate.
6. The process of any of embodiments 1 to 5, wherein independently from one another, the one or more silicon containing compounds in (ii) and (iv) are selected from the group consisting of silicates, preferably from the group consisting of silicate salts, more preferably from the group consisting of alkali metal silicates and mixtures thereof, wherein the alkali metal is preferably selected from the group consisting of Li, Na, K, Rb, Cs, and mixtures of two or more thereof, more preferably from the group consisting of Li, Na, K, and mixtures of two or more thereof, wherein more preferably the alkali metal is Na and/or K, preferably Na,
   and wherein more preferably the one or more silicon containing compounds comprise sodium silicate, preferably sodium water glass, and more preferably Na₂SiO₃, wherein more preferably the one or more silicon containing compounds is sodium silicate, preferably sodium water glass, and more preferably Na₂SiO₃.
7. The process of any of embodiments 1 to 6, wherein in (i), Cu, Zn, Al, and Zr are respectively contained in S1 as salts, preferably as nitrate salts.
8. The process of any of embodiments 1 to 7, wherein the solution S1 obtained from (i) displays a Cu:Zn:Al:Zr molar ratio in the ranges of (40-65):(10-25):(15-40):(0.2-10), preferably of (45-62):(13-23):(18-35):(0.5.-5), more preferably of (50-59):(15-21):(22-30):(1-3), and more preferably of (55-56):(17-19):(25-26):(1.4-1.6).
9. The process of any of embodiments 1 to 8, wherein in (iii) the pH of the aqueous mixture M is kept in the pH range by metering a third aqueous solution S3 into the aqueous mixture M, wherein the pH of S3 is equal to or higher than the pH of S2, wherein the pH of S3 is preferably in the range of from 11 to 14, more preferably of from 12 to 14, and more preferably of from 13 to 14.
10. The process of embodiment 9, wherein S3 comprises one or more bases selected from the group consisting of Bronstedt and Lewis bases, wherein preferably the one or more bases are selected from the group consisting of inorganic and organic bases, more preferably from the group of inorganic bases, wherein preferably the one or more bases are selected from the group consisting of hydroxides, carbonates, aluminates, and mixtures of two or more thereof,
   more preferably from the group consisting of alkali metal hydroxides, alkali metal carbonates, alkali metal aluminates, and mixtures of two or more thereof,
   more preferably from the group consisting of alkali metal carbonates, alkali metal aluminates, and mixtures of two or more thereof,
   wherein the alkali metal is preferably selected from the group consisting of Li, Na, K, Rb, Cs, and mixtures of two or more thereof,
   more preferably from the group consisting of Li, Na, K, and mixtures of two or more thereof,
   wherein more preferably the alkali metal is Na and/or K, preferably Na,
   and wherein more preferably the one or more bases comprise sodium carbonate and/or sodium hydroxide, preferably sodium carbonate and sodium hydroxide, wherein more preferably the one or more bases are sodium carbonate and/or sodium hydroxide, preferably sodium carbonate and sodium hydroxide.
11. The process of any of embodiments 1 to 10, wherein adding in (iii) is conducted continuously or intermittently, preferably continuously.
12. The process of any of embodiments 1 to 11, wherein for keeping the pH of the aqueous mixture M in the pH range in (iii), the pH of the aqueous mixture M is semi-continuously or continuously monitored, preferably continuously monitored.
13. The process of any of embodiments 1 to 12, wherein agitation in (iii) is achieved by stirring.
14. The process of any of embodiments 1 to 13, wherein aging in (iv) is conducted at a temperature in the range of from 5 to 75° C., preferably of from 15 to 70° C., more preferably of from 25 to 65° C., more preferably of from 40 to 60° C., and more preferably of from 45 to 55° C.
15. The process of any of embodiments 1 to 14, wherein in (iv) the sodium water glass is added after a period of aging in the range of from 0.5-11.5 h, wherein preferably the sodium water glass is optionally added after 1 h of aging.
16. The process of any of embodiments 1 to 15, wherein the aqueous mixture M obtained from (iii) or the aged aqueous mixture obtained from (iv) displays a Cu:Zn:Al:Zr:Si molar ratio in the ranges of (40-65):(10-25):(15-40):(0.2-10):(0.1-5), preferably of (45-62):(13-23):(18-35):(0.5.-5):(0.3-4.5), more preferably of (50-59):(15-21):(22-30):(1-3):(0.8-4), and more preferably of (55-56):(17-19):(25-26):(1.4-1.6):(1.2-3.6).
17. The process of any of embodiments 1 to 16, wherein separation in (v) is achieved by filtration.
18. The process of any of embodiments 1 to 17, wherein the process further comprises:
   (vi) washing the solid obtained from (v); and/or, preferably and
   (vii) drying the solid obtained from (v) or (vi), preferably at a temperature in the range of from 80 to 150° C., preferably of from 90 to 140° C., more preferably of from 105 to 130° C., and more preferably of from 115 to 125° C.
19. The process of embodiment 18, wherein washing in (vi) is performed with deionized water.
20. The process of embodiment 18 or 19, wherein drying in (vii) is conducted for a duration in the range of from 1 to 48 h, preferably of from 6 to 36 h, more preferably of from 12 to 30 h, and more preferably of from 18 to 24 h.
21. The process of any of embodiments 1 to 20, wherein the process further comprises:
   (ix) calcining the solid obtained from (v), (vi), or (vii), preferably at a temperature in the range of from 400 to 750° C., preferably of from 500 to 700° C., and more preferably of from 550 to 650° C.
22. The process of embodiment 21, wherein calcining in (ix) is conducted for a duration in the range of from 0.5 to 12 h, preferably from 1 to 6 h, and more preferably from 1.5 to 2.5 h.
23. The process of embodiment 21 or 22, wherein calcining in (ix) is performed in an atmosphere containing oxygen, preferably in an atmosphere containing air, wherein more preferably calcining in (ix) is performed in air.
24. A catalyst precursor comprising Cu, Zn, Al, Zr, Si, and O, preferably according to any of embodiments 31 to 39, as obtainable or obtained according to the process of any of embodiments 1 to 23.
25. A process for the preparation of a supported copper catalyst comprising Cu, Zn, Al, Zr, Si, and O, preferably of a supported copper catalyst according to any of embodiments 40 to 46, wherein the catalyst comprises elemental copper, the process comprising:
   (1) preparing a catalyst precursor comprising Cu, Zn, Al, Zr, Si, and O, according to the process of any of embodiments 21 to 23;
   (2) reducing the catalyst precursor obtained from (1) in an atmosphere comprising hydrogen for obtaining the supported copper catalyst.
26. The process of embodiment 25, wherein reduction in (2) is conducted at a temperature in the range of from 150 to 350° C., preferably of from 170 to 300° C., and more preferably of from 170 to 230° C.
27. The process of embodiment 25 or 26, wherein the atmosphere in (2) comprises from 0.25 to 80 vol.-% H₂, preferably from 0.5 to 50 vol.-% H₂, more preferably from 0.5 to 30 vol.-% H₂, more preferably from 1 to 10 vol.-% H₂, and more preferably from 2 to 5 vol.-% H₂.
28. The process of any of embodiments 25 to 27, wherein the atmosphere in (2) comprises from 99.75 to 20 vol.-% of an inert gas, preferably from 99.5 to 50 vol.-%, more preferably from 99.5 to 70 vol.-%, more preferably from 99 to 90 vol.-%, and more preferably from 98 to 95 vol.-%.
29. The process of embodiment 28, wherein the inert gas comprises one or more gases selected from the group consisting of noble gases, nitrogen gas, and methane, preferably from the group consisting of He, Ar, Ne, N₂, and CH4, wherein more preferably the inert gas comprises N₂, wherein more preferably the inert gas is N₂.
30. A supported copper catalyst comprising Cu, Zn, Al, Zr, Si, and O, preferably according to any of embodiments 40 to 46, as obtainable or obtained according to the process of any of embodiments 25 to 29, wherein the catalyst comprises elemental copper.
31. A catalyst precursor comprising Cu, Zn, Al, Zr, Si, and O, preferably as obtainable or obtained according to the process of embodiments 1 to 23, wherein the catalyst precursor displays a Zn:Si atomic ratio in the range of from 5:1 to 27:1, preferably of from 5.5:1 to 25:1, more preferably of from 6:1 to 20:1, more preferably of from 6.5:1 to 15:1, more preferably of from 7:1 to 12:1, more preferably of from 7.5:1 to 10:1, and more preferably of from 8:1 to 9:1.
32. The catalyst precursor of embodiment 31, wherein the catalyst precursor comprises one or more hydroxycarbonate mixed oxides comprising two or more of Cu, Zn, and Al, preferably two or more of Cu, Zn, Al, and Zr, wherein more preferably the catalyst precursor comprises one or more hydroxycarbonate mixed oxides of Cu, Zn, and Al, preferably of Cu, Zn, Al, and Zr.
33. The catalyst precursor of embodiment 31 or 32, wherein the catalyst precursor comprises CuO.
34. The catalyst precursor of any of embodiments 31 to 33, wherein the catalyst precursor comprises Zr-modified Cu₃Zn₃Al₂(OH)₁₆CO₃, preferably a Zr-modified Cu₃Zn₃Al₂(OH)₁₆CO₃·4H₂O, wherein the 003 reflection in the x-ray diffractogram of the Zr-modified Cu₃Zn₃Al₂(OH)₁₆CO₃ is shifted to higher ° 2Theta values compared to the x-ray diffractogram of Cu₃Zn₃Al₂(OH)₁₆CO₃, wherein the 003 reflection in the x-ray diffractogram of the Zr-modified Cu₃Zn₃Al₂(OH)₁₆CO₃ is located in the range of from 11 to 13.5° 2Theta, wherein the x-ray diffractogram is preferably determined according to Reference Example 2.
35. The catalyst precursor of embodiment 31, preferably as obtainable or obtained according to the process of embodiments 21 to 23, wherein the catalyst precursor comprises one or more oxides of Cu, Zn, Al, Zr, and Si, wherein the catalyst precursor preferably comprises one or more oxides selected from the group consisting of CuO, ZnO, ZnAl₂O₄, and CuAl₂O₄, wherein more preferably the catalyst precursor comprises CuO, ZnO, and ZnAl₂O₄, or CuO, ZnO, and CuAl₂O₄, or CuO, ZnO, ZnAl₂O₄, and CuAl₂O₄.
36. The catalyst precursor of embodiment 35, wherein the catalyst precursor comprises CuO in an amount in the range of from 50 to 70 wt.-% calculated as the oxide CuO and based on the sum of the weights of the oxides of Cu, Zn, Al, Zr, and Si contained in the catalyst precursor, calculated as CuO, ZnO, Al₂O₃, ZrO₂, and SiO₂, wherein preferably, the catalyst precursor comprises CuO in an amount in the range of from 55 to 65 wt. %, and more preferably of from 58 to 62 wt.-%.
37. The catalyst precursor of embodiment 35 or 36, wherein the catalyst precursor comprises SiO₂.
38. The catalyst precursor of any of embodiments 31 to 37, wherein the catalyst precursor displays a Cu:Zn:Al:Zr:Si molar ratio in the ranges of (40-65):(10-25):(15-40):(0.2-10):(0.1-5), preferably of (45-62):(13-23):(18-35):(0.5.-5):(0.3-4.5), more preferably of (50-59):(15-21):(22-30):(1-4):(0.8-4), and more preferably of (55-56):(17-19):(25-26):(1.4-3):(1.2-3.6).
39. The catalyst precursor of any of embodiments 31 to 38, wherein from 95 to 100 wt.-% of the catalyst precursor consists of Cu, Zn, Al, Zr, Si, and O, preferably from 97 to 100 wt.-%, more preferably from 98 to 100 wt.-%, more preferably from 99 to 100 wt.-%, more preferably from 99.5 to 100 wt.-%, and more preferably from 99.9 to 100 wt.-%.
40. A supported copper catalyst comprising Cu, Zn, Al, Zr, Si, and O, preferably as obtainable or obtained according to the process of embodiments 25 to 29, wherein the catalyst comprises elemental copper, and wherein the catalyst displays a Zn:Si atomic ratio in the range of from 5:1 to 27:1, preferably of from 5.5:1 to 25:1, more preferably of from 6:1 to 20:1, more preferably of from 6.5:1 to 15:1, more preferably of from 7:1 to 12:1, more preferably of from 7.5:1 to 10:1, and more preferably of from 8:1 to 9:1.
41. The supported copper catalyst of embodiment 40, wherein the supported copper catalyst comprises one or more oxides of Zn, Al, Zr, and Si, wherein the supported copper catalyst preferably comprises one or more oxides selected from the group consisting of ZnO, ZnAl₂O₄, and CuAl₂O₄, wherein more preferably the supported copper catalyst comprises ZnO and ZnAl₂O₄, or ZnO and CuAl₂O₄, or ZnO, ZnAl₂O₄, and CuAl₂O₄.
42. The supported copper catalyst of embodiment 40 or 41, wherein the supported copper catalyst displays a BET surface area of 130 m²/g or less, and preferably displays a BET surface area within the range of from 60 to 130 m²/g, wherein the BET surface area is preferably determined according to Reference Example 1.
43. The supported copper catalyst of any of embodiments 40 to 42, wherein the supported copper catalyst displays an copper surface area in the range of from 5 to 15 m²/g, preferably for from 10 to 13 m²/g,
   wherein the copper surface area is determined according to Reference Example 3.
44. The supported copper catalyst of any of embodiments 40 to 43, wherein the supported copper catalyst comprises SiO₂.
45. The supported copper catalyst of any of embodiments 40 to 44, wherein the supported copper catalyst displays a Cu:Zn:Al:Zr:Si molar ratio in the ranges of (40-65):(10-25):(15-40):(0.2-10):(0.1-5), preferably of (45-62):(13-23):(18-35):(0.5.-5):(0.3-4.5), more preferably of (50-59):(15-21):(22-30):(1-4):(0.8-4), and more preferably of (55-56):(17-19):(25-26):(1.4-3):(1.2-3.6).
46. The supported copper catalyst of any of embodiments 40 to 45, wherein from 95 to 100 wt.-% of the supported copper catalyst consists of Cu, Zn, Al, Zr, Si, and O, preferably from 97 to 100 wt.-%, more preferably from 98 to 100 wt.-%, more preferably from 99 to 100 wt.-%, more preferably from 99.5 to 100 wt.-%, and more preferably from 99.9 to 100 wt.-%.
47. The supported copper catalyst of any of embodiments 40 to 46, being in the form of a molding, wherein the molding has a height H and a tristar cross-section with rounded tips and with rounded corners.
48. The supported copper catalyst of any of embodiment 47, wherein the tip basis b of the tristar cross-section with rounded tips and with rounded corners is in the range of from 2.00 to 2.60 mm, preferably in the range of from 2.10 to 2.50 mm, more preferably in the range of from 2.20 to 2.40 mm, more preferably in the range of from 2.25 to 2.35 mm.
49. The supported copper catalyst of any of embodiment 47 or 48, wherein the tip height i of the tristar cross-section with rounded tips and with rounded corners is in the range of from 2.55 to 3.15 mm, preferably in the range of from 2.65 to 3.05 mm, more preferably in the range of from 2.75 to 2.95 mm, more preferably in the range of from 2.80 to 2.90 mm.
50. The supported copper catalyst of any of embodiments 47 to 49, wherein the height h of the tristar cross-section with rounded tips and with rounded corners is in the range of from 5.40 to 6.00 mm, preferably in the range of from 5.50 to 5.90 mm, more preferably in the range of from 5.60 to 5.80 mm, more preferably in the range of from 5.65 to 5.75 mm.
51. The supported copper catalyst of any of embodiments 47 to 50, wherein the chord length k of the tristar cross-section with rounded tips and with rounded corners is in the range of from 6.00 to 6.60 mm, preferably in the range of from 6.10 to 6.50 mm, more preferably in the range of from 6.20 to 6.40 mm, more preferably in the range of from 6.25 to 6.35 mm.
52. The supported copper catalyst of any of embodiments 47 to 51, wherein the tristar cross-section with rounded tips and with rounded corners has an inner angle α in the range of from 5 to 19°, preferably in the range of from 8 to 16°, more preferably in the range of from 10 to 14°, more preferably in the range of from 11 to 13°.
53. The supported copper catalyst of any of embodiments 47 to 52, wherein each of the tips of the tristar cross-section independently from each other is rounded by an arc of a circle having a radius in the range of from 0.65 to 1.25 mm, preferably in the range of from 0.75 to 1.15 mm, more preferably in the range of from 0.85 to 1.05 mm, more preferably in the range of from 0.90 to 1.00 mm.
54. The supported copper catalyst of any of embodiments 47 to 53, wherein the distance between the geometric center C of the tristar cross-section and each of the tips of the tristar cross-section independently from each other is in the range of from 2.0 to 5.0 mm, preferably in the range of from 2.7 to 4.3 mm, more preferably in the range of from 3.2 to 3.8 mm, more preferably in the range of from 3.4 to 3.6 mm.
55. The supported copper catalyst of any of embodiments 47 to 54, wherein the tristar cross-section with rounded tips and with rounded corners has an outer angle θ in the range of from 125 to 155°, preferably in the range of from 135 to 145°, more preferably in the range of from 127 to 137°, more preferably in the range of from 130 to 134°.
56. The supported copper catalyst of any of embodiments 47 to 55, wherein each of the corners of the tristar cross-section independently from each other is rounded by an arc of a circle having a radius in the range of from 0.65 to 1.25 mm, preferably in the range of from 0.75 to 1.15 mm, more preferably in the range of from 0.85 to 1.05 mm, more preferably in the range of from 0.90 to 1.00 mm.
57. The supported copper catalyst of any of embodiments 47 to 56, wherein the height H of the molding is in the range of from 3.0 to 11.0 mm, preferably in the range of from 3.5 to 10.5 mm, more preferably in the range of from 4.5 to 9.5 mm, more preferably in the range of from 5.5 to 8.5 mm, more preferably in the range of from 6.5 to 7.5 mm.
58. A method for the conversion of CO₂-containing syngas to methanol, the method comprising:
   (A) providing a supported copper catalyst according to any of embodiments 30 or 40 to 46;
   (B) preparing a gas mixture comprising CO, H₂, and CO₂;
   (C) contacting the supported copper catalyst provided in (A) with the gas mixture prepared in (B), wherein the contacting is performed at a temperature in the range of from 200 to 350° C., preferably of from 230 to 260° C.
59. The method of embodiment 58, wherein contacting in (B) is performed at a pressure in the range of from 1 to 100 bara, preferably in the range of from 40 to 85 bara, more preferably from 70 to 82 bara, and more preferably from 74 to 81 bara.
60. The method of embodiment 58 or 59, wherein the gas mixture prepared in (B) comprises from 10 to 24 vol.-% CO₂, preferably from 11 to 20 vol.-%, more preferably from 12 to 19 vol.-%, and more preferably from 15 to 18 vol.-%.
61. The method of any of embodiments 58 to 60, wherein the gas mixture prepared in (B) comprises from 0.5 to 7 vol.-% CO, preferably from 0.8 to 4 vol.-%, and more preferably from 1 to 2 vol.-%.
62. The method of any of embodiments 58 to 61, wherein the gas mixture prepared in (B) displays a CO₂:CO molar ratio in the range of from 2 to 20, preferably for from 3 to 17, more preferably for from 5 to 15, and more preferably for from 7 to 13.
63. The method of any of embodiments 58 to 62, wherein the gas mixture prepared in (B) comprises from 50 to 90 vol.-% H₂, preferably from 55 to 87 vol.-%, more preferably from 60 to 85 vol.-%, and more preferably from 65 to 83 vol.-%.
64. The method of any of embodiments 58 to 63, wherein the gas mixture prepared in (B) comprises from 0.1 to 40 vol.-% of an inert gas, preferably from 0.3 to 30 vol.-%, more preferably from 0.5 to 25 vol.-%, more preferably from 0.8 to 20 vol.-%, and more preferably from 1 to 15 vol.-%.
65. The method embodiment 64, wherein the inert gas comprises one or more gases selected from the group consisting of noble gases and nitrogen gas, preferably from the group consisting of He, Ar, Ne, CH₄, and N₂, more preferably from the group consisting of Ar, CH₄, N₂, wherein more preferably the inert gas comprises CH₄ and N₂, wherein more preferably the inert gas is CH₄ and N₂.
66. A method for the conversion of syngas to dimethyl ether, the method comprising:
   (A) mixing a supported copper catalyst according to any of embodiments 30 or 40 to 57 with an acidic co-catalyst;
   (B) preparing a gas mixture comprising CO, H₂, and CO₂;
   (C) contacting the catalyst mixture prepared in (A) with the gas mixture prepared in (B), wherein the contacting is performed at a temperature in the range of from 200 to 300° C., preferably of from 250 to 270° C.
67. The method of embodiment 66, wherein contacting in (B) is performed at a pressure in the range of from 1 to 100 bara, preferably in the range of from 50 to 85 bara, more preferably of from 60 to 75 bara.
68. The method of embodiment 66 or 67, wherein the gas mixture prepared in (B) comprises 25 vol.-% or less of CO₂, preferably from 2 to 15 vol.-% CO₂, more preferably from 3 to 10 vol.-%, and more preferably from 4 to 6 vol.-%.
69. The method of any of embodiments 66 to 68, wherein the gas mixture prepared in (B) comprises from 2 to 30 vol.-% CO, preferably from 5 to 29 vol.-%, more preferably from 10 to 28 vol.-%, more preferably from 20 to 27 vol.-%, and more preferably from 24 to 26 vol.-%.
70. The method of any of embodiments 66 to 69, wherein the gas mixture prepared in (B) displays a CO₂:CO molar ratio of 5 or less, preferably a CO₂:CO molar ratio in the range of from 0.05 to 2, more preferably in the range of from 0.05 to 1, more preferably of from 0.1 to 0.5, and more preferably of from 0.15 to 0.25.
71. The method of any of embodiments 66 to 70, wherein the gas mixture prepared in (B) comprises from 30 to 70 vol.-% H₂, preferably from 40 to 65 vol.-%, and more preferably from 50 to 60 vol.-%.
72. The method of any of embodiments 66 to 71, wherein the gas mixture prepared in (B) comprises from 1 to 30 vol.-% of an inert gas, preferably from 5 to 25 vol.-%, and more preferably from 10 to 20 vol.-%.
73. The method of embodiment 72, wherein the inert gas comprises one or more gases selected from the group consisting of noble gases and nitrogen gas, preferably from the group consisting of He, Ar, Ne, CH₄, and N₂, wherein more preferably the inert gas comprises CH₄ and N₂, wherein more preferably the inert gas is N₂.
74. Use of a supported copper catalyst according to any of embodiments 30 or 40 to 57 as a reverse water-gas shift catalyst, in the reforming of methanol, in the conversion of CO₂-containing syngas to methanol, in the reforming of dimethyl ether, and in the conversion of syngas to dimethyl ether.

The present invention is further illustrated by the following Reference Examples, Examples, and Comparative Examples.

### EXPERIMENTAL SECTION

The samples were synthesized in a stirred reaction vessel under full control of temperature, pH and dosing of the solutions.

### Reference Example 1: Determination of BET Surface Area

The BET specific surface area was determined via nitrogen physisorption at 77 K according to the method disclosed in DIN 66131.

### Reference Example 2: X-Ray Powder Diffraction and Determination of Crystallinity

Powder X-ray diffraction (PXRD) data was collected using a diffractometer (D8 Advance Series II, Bruker AXS GmbH) equipped with a LYNXEYE detector operated with a Copper anode X-ray tube running at 40 kV and 40 mA. The geometry was Bragg-Brentano, and air scattering was reduced using an air scatter shield.

Computing crystallinity: The crystallinity of the samples was determined using the software DIFFRAC.EVA provided by Bruker AXS GmbH, Karlsruhe, according to the method which is described on page 121 of the user manual. The default parameters for the calculation were used.

Computing phase composition: The phase composition was computed against the raw data using the modelling software DIFFRAC.TOPAS provided by Bruker AXS GmbH (User Manual for DIFFRAC.TOPAS Version 6, 2017, Bruker AXS GmbH, Karlsruhe). The crystal structures of the identified phases, instrumental parameters as well the crystallite size of the individual phases were used to simulate the diffraction pattern. This was fit against the data in addition to a function modelling the background intensities.

Data collection: The samples were homogenized in a mortar and then pressed into a standard flat sample holder provided by Bruker AXS GmbH for Bragg-Brentano geometry data collection. The flat surface was achieved using a glass plate to compress and flatten the sample powder. The data was collected from the angular range 2 to 70° 2Theta with a step size of 0.02° 2Theta, while the variable divergence slit was set to an angle of 0.1°. The crystalline content describes the intensity of the crystalline signal to the total scattered intensity.

### Reference Example 3: Determination of Cu Surface Area

The Cu surface area was determined according to the method disclosed in EP 0202824 A using N₂O and the pulse method at 25° C.

### Reference Example 4: Overview of Prepared Catalysts

TABLE 1
Overview of the compositions of the acidic metal solutions based on the
corresponding metal oxides in weight-%. The contents are noted as oxides
in weight-% with respect to the calcined powder prior to addition of
a binder, shaping process and activation under reductive conditions.

| | SiO₂ addition | | | | | | Temp. |
|---|---|---|---|---|---|---|---|
| | according to | CuO | ZnO | Al₂O₃ | ZrO₂ | SiO₂ | ° C. |
| # | example | [wt.-%] | [wt.-%] | [wt.-%] | [wt.-%] | [wt.-%] | co-prec. |
| Ex. 1 | no SiO₂ | 60 | 20 | 17.5 | 2.5 | 0 | 14 |
| Ex. 2 | I | 60 | 20 | 17.5 | 2.4 | 0.1 | 30 |
| Ex. 3 | I | 60 | 20 | 17.5 | 2.3 | 0.2 | 30 |
| Ex. 4 | I | 60 | 20 | 17.5 | 2 | 0.5 | 30 |
| Ex. 5 | I | 60 | 20 | 17.5 | 1.5 | 1 | 30 |
| Ex. 6 | I | 60 | 18.2 | 15.8 | 3.7 | 2.3 | 50 |
| Ex. 7 | II | 60 | 18.9 | 12.5 | 6.9 | 1.7 | 14 |
| Ex. 8 | II | 60 | 15.8 | 17.5 | 3.7 | 3 | 28 |
| Ex. 9 | no SiO₂ | 60 | 16.5 | 11.7 | 11.8 | 0 | 39 |
| Ex. 10 | I | 60 | 22 | 10 | 5.3 | 2.7 | 28 |
| Ex. 11 | II | 60 | 20.4 | 14.2 | 3.7 | 1.7 | 39 |
| Ex. 12 | I | 60 | 15.7 | 15 | 8.6 | 0.7 | 28 |
| Ex. 13 | no SiO₂ | 60 | 15.8 | 10.8 | 13.4 | 0 | 28 |
| Ex. 14 | no SiO₂ | 60 | 15.8 | 10.8 | 13.4 | 0 | 18 |
| Ex. 15 | II | 60 | 22 | 10 | 5.3 | 2.7 | 28 |
| Ex. 16 | I | 60 | 20.4 | 14.2 | 3.7 | 1.7 | 18 |
| Ex. 17 | I | 60 | 18.9 | 12.5 | 6.9 | 1.7 | 50 |
| Ex. 18 | II | 60 | 16.6 | 15.8 | 6.9 | 0.7 | 18 |
| Ex. 19 | no SiO₂ | 60 | 18.1 | 13.3 | 8.6 | 0 | 28 |
| Ex. 20 | no SiO₂ | 60 | 20.5 | 15.8 | 3.7 | 0 | 39 |
| Ex. 21 | II | 60 | 18 | 11.7 | 8.6 | 1.7 | 39 |
| Ex. 22 | I | 60 | 15 | 16.7 | 5.3 | 3 | 18 |
| Ex. 23 | I | 60 | 18 | 11.7 | 8.6 | 1.7 | 18 |
| Ex. 24 | no SiO₂ | 60 | 21.2 | 16.7 | 2.1 | 0 | 13 |
| Ex. 25 | II | 60 | 16.6 | 15.8 | 6.9 | 0.7 | 50 |

### Example I: Preparation of a Supported Cu Catalyst Comprising CuO/ZnO/Al₂Od/ZrO₂/SiO₂ Wherein the SiO₂ Source is Added in the Precipitation Reactor Prior to Dosing

Demineralized water (1000 g) was filled into a reaction vessel and brought to the target temperature (between 15 and 45° C.). To the demineralized water, the proper amount of natron water glass (between 1 and 60 g of an aqueous solution with 26 weight-% Si calculated as SiO₂) was added. The pH was adjusted to 9 and the co-precipitation was started. Under continuous stirring, an acidic metal nitrate solution of Cu, Zn, Al and Zr (3,100 g, density 1.4 kg/l, composition see Table 1) was dosed. In parallel, an aqueous base solution (11,000 g) of NaOH (2 M) mixed with Na₂CO₃(0.3 M) was added to control the pH value to be maintained at 9. During the entire co-precipitation process, the pH and the temperature were kept constant. After the complete dosing of the solutions, the resulting suspension was aged for 2 hours at 50° C. under stirring. Afterwards, the suspension was filtered and the residual solid (700 to 800 g) was washed. The washed solid was dried over night at 120° C. to finally obtain a catalyst precursor. The precursor was calcined at 600° C. under synthetic air (21 volume-% O₂/79 volume-% N₂). The resulting metal oxides powder was mixed with a graphitic binder and then tableted.

### Example II: Preparation of a Supported Cu Catalyst Comprising CuO/ZnO/Al₂O/ZrO₂/SiO₂ Wherein the SiO₂ Source is Added During the Aging

Demineralized water (1000 g) was filled into a reaction vessel and brought to the target temperature (between 15 and 45° C.). The pH was adjusted to 9 and the co-precipitation was started. Under continuous stirring, an acidic metal nitrate solution of Cu, Zn, Al and Zr (3100 g, density of 1.4 kg/l, composition see Table 1) was dosed. In parallel, an aqueous base solution (11,000 g) of NaOH (2 M) mixed with Na₂CO₃(0.3 M) was added to control the pH value to be maintained at 9. During the entire co-precipitation process, the pH and the temperature were kept constant. After the complete dosing of the solutions, the resulting suspension was aged for 2 hours at 50° C. under stirring. After 1 hour of aging, the proper amount of natron water glass was added (between 1 and 60 g of an aqueous solution with 26 weight-% Si calculated as SiO₂). Afterwards, the suspension was filtered and the residual solid (700 to 800 g) was washed. The washed solid was dried over night at 120° C. to finally obtain a catalyst precursor. The precursor was calcined at 600° C. under synthetic air (21 volume-% O₂/79 volume-% N₂. The resulting metal oxides powder was mixed with a graphitic binder and then tableted.

### Example III: Catalyst Testing in the Methanol Synthesis from CO₂, CO, and H₂

First, the catalysts were activated in a reductive atmosphere (5% H₂ in Ar) and a temperature of up to 250° C. Once the activation was completed, the pressure was increased to 80 bara and the testing protocol was started. The tested CO₂-rich syngas consisted of 15 volume-% CO₂, 2 volume-% CO, 73 volume-% H₂, rest N₂ and was fed at a gas hourly space velocity (GHSV) in the range of from 6000 to 12000 h⁻¹. A content of 2 volume-% of CO was chosen for simulating a process wherein a feed stream of H₂ and CO₂ would be applied as make-up-gas and wherein CO would be accumulating in the recycle due to reverse water-gas shift contributions as side reaction (see equation 3).

In between reference points set at 100 and 200 h time on stream (TOS), a rapid aging step (dwell for 36 h) was included, which was defined as rich in CO₂ (30 volume-%) under-stoichiometric in H₂ (60 volume-%), rest N₂, at 260° C. and a GHSV of 12000 h⁻¹. The reference point after the rapid aging experiment was crucial to monitor the deactivation in particular the stability of the used catalyst. The catalysts were tested as sieve fractions of 400 to 500 microns.

Table 2 shows the relative space-time-yield (STY) values, coupled to the ZnO:SiO₂ weight ratio and the difference in activity before and after the rapid aging in %. Negative values correspond to deactivation, positive values to activation. In addition, the N₂O surface area is listed (determination method according to EP 0202824 A, pulse method at 25° C.).

TABLE 2
Results from catalytic testing using a highly CO₂-enriched syngas
shown as relative space-time-yield and activity difference after
100 and 200 h time on stream. Further, characteristics of
used catalyst including N₂O surface area values and ZnO:SiO₂
weight ratio are shown for the prepared Examples.

| | SiO₂ | | | Activity | |
|---|---|---|---|---|---|
| | addition | ZnO: | relative | diff. | SA in m²/ |
| | according | SiO₂ | STY | after 100 | g from |
| | to | [wt.: | [gMeOH/ | and 200 h | N₂O- |
| Example | Example | wt.] | (mlcath)] | TOS [%] | pulsing |
| Comp. Ex. 1 | (no Si) | — | 1.57 | −17 | 12.7 |
| Comp. Ex. 9 | (no Si) | — | 1.56 | −12 | 12.32 |
| Comp. Ex. 13 | (no Si) | — | 1.62 | −12 | 11.76 |
| Comp. Ex. 14 | (no Si) | — | 1.71 | −12 | 11.79 |
| Comp. Ex. 19 | (no Si) | — | 1.69 | −12 | 11.45 |
| Comp. Ex. 20 | (no Si) | — | 1.54 | −18 | 11.95 |
| Comp. Ex. 24 | (no Si) | — | 1.60 | −14 | 11.87 |
| Comp. Ex. 2 | I | 200.0 | 1.50 | −14 | 11.4 |
| Comp. Ex. 3 | I | 100.0 | 1.61 | −12 | 10.96 |
| Comp. Ex. 4 | I | 40.0 | 1.56 | −8 | 10.3 |
| Ex. 18 | II | 23.7 | 1.46 | −6 | 9.88 |
| Ex. 25 | II | 23.7 | 1.57 | −8 | 11.18 |
| Ex. 12 | I | 22.4 | 1.47 | −6 | 9.98 |
| Ex. 5 | I | 20.0 | 1.67 | −4 | 10.18 |
| Ex. 11 | II | 12.0 | 1.52 | −4 | 9.98 |
| Ex. 16 | I | 12.0 | 1.79 | −5 | 9.49 |
| Ex. 7 | II | 11.1 | 1.72 | −3 | 9.58 |
| Ex. 17 | I | 11.1 | 1.62 | −4 | 11.63 |
| Ex. 21 | II | 10.6 | 1.48 | −5 | 11.18 |
| Ex. 23 | I | 10.6 | 1.61 | −3 | 10.56 |
| Ex. 10 | I | 8.1 | 1.21 | 9 | 9.9 |
| Ex. 15 | II | 8.1 | 1.28 | 1 | 10.76 |
| Ex. 6 | I | 7.9 | 1.41 | 2 | 10.07 |
| Comp. Ex. 22 | I | 5.0 | 1.00 | 29 | 8.03 |
| Comp. Ex. 8 | II | 5.3 | 1.10 | 15 | 9.7 |

As it can be gathered from the results shown in table 2, the catalysts in accordance with the present invention, thus having a specific Zn to Si atomic ratio, show a very good spacetime-yield, as well as a comparatively low activity decrease.

### Example IV: Catalyst Testing in a Direct Synthesis of Dimethyl Ether from CO₂, CO, and H₂

The catalyst according to Example 16 was applied in a one-step DME synthesis, wherein the catalyst was mixed with an acidic co-catalyst (zeolite-type catalyst according to EP 3727681 A1) for the dehydration of the MeOH to DME. The activation protocol was identical to that according to Example III. The experiment was conducted at 63 bara, a temperature between 220 to 280° C., whereby a CO-rich syngas (50 volume-% H₂, 25 volume-% CO, 5 volume-% CO₂, rest N₂) was fed at a GHSV of 3000 h⁻¹.

The results of the catalytic testing are shown in FIGS. 4A and 4B. As it can be gathered from FIG. 4A the catalyst according to Example 16 showed a high CO conversion of slightly below 90%. After starting the process the CO conversion constantly increased during the process up to about 92%.

As it can be gathered from FIG. 4B the catalyst according to Example 16 showed a high selectivity towards dimethyl ether. In particular, the selectivity towards dimethyl ether was about 63% shortly after starting the process and increased slightly up to about 64%. Further, the selectivity towards CO₂ was only about 27% shortly after starting the process and decreased to 26%, whereas the selectivity towards MeOH was about 8% shortly after starting the process and increased up to about 10%. All in all, this catalyst activates over a long TOS and improves its selectivity towards DME.

In sum, it was found that the catalyst according to Example 16 showed a very good performance indicated by stable conversions of CO and CO₂ as well as by stable selectivities towards dimethyl ether, CO₂ and MeOH. Therefore, it was shown that the catalyst according to Example 16 represents an improved catalyst, especially showing good testing results under demanding conditions being a temperature in the range of from 220 to 280° C. and a pressure of 63 bara. Also, it was shown that the catalyst according to Example 16 can withstand high partial pressures of CO₂ and H₂O. Accordingly, the invented catalysts are applicable in a CO₂ or CO₂-enriched syngas hydrogenation reaction where a high partial pressure of CO₂ is part of the reactor feed (see equations 2 and 3) or H₂O is specifically enriched as by-product (see equation 4).

### Example V: Evaluation of Pressure Drop for Tablets and Moldings

A CFD simulation was performed for evaluating the influence of the geometry of two different moldings on the backpressure.

For a geometry representing the prior art, tablets having a diameter of 6 mm and a height of 4 mm were used for the simulation. Said tablets represent moldings which could be prepared by mixing a metal oxides powder with a graphitic binder and then tableting.

For a geometry according to the present invention, moldings having a tristar cross-section, wherein the tips are rounded and wherein the corners are rounded, were used for the simulation. Said moldings could be prepared by mixing a metal oxides powder according to the present invention with a graphitic binder and forming to moldings.

A catalytic test was performed by a CFD simulation for the tablets as well as for the moldings.

The pressure drop A p in Pa/m and the relative pressure drop were calculated as follows:

$$Δ p = ( {p}_{in} - {p}_{out} ) / H , and$$

$$relative Δ p = Δ p / Δ {p}_{ref} .$$

It was found that the pressure drop of the tablets was 1, wherein it was 0.55 for the moldings.

As a result, it was found that said moldings allow for a considerably higher gas hourly space velocity at the same level of backpressure.

### DESCRIPTION OF FIGURES

FIGS. 1A and 1B: In FIG. 1A the powder XRD is shown of the dried precipitate according to Example 16 obtained after co-precipitation and drying, but before calcination. The characteristic hydrotalcite phase was identified therein.

FIG. 1B shows the powder XRD of the dried precipitate according to Example 12 obtained after co-precipitation and drying, but before calcination

FIG. 2: shows the powder XRD of the calcined catalyst according to Example 16. It is shown that besides the CuO and ZnO phases also the ZnAl₂O₄ spinel phase is formed from the hydrotalcite precursor.

FIG. 3: shows the powder XRD of the calcined and activated catalyst according to Example 8. It is shown that besides the Cu metal, a typical ZnAl₂O₄ spinel phase is formed from the hydrotalcite precursor. Further, the existence of CuAl₂O₄, in addition to the ZnAl₂O₄, is likely.

FIGS. 4A and 4B: FIG. 4A shows the results for the catalytic testing according to Example IV with respect to the one-step dimethyl ether.

FIG. 5: shows a schematic of a molding having a tristar cross-section, wherein the tristar cross-section has a geometric center C, and wherein each of the tips of the tristar cross-section is rounded by an arc of a circle, and wherein each of the corners of the tristar cross-section is rounded by an arc of a circle.

FIG. 6: shows a schematic of a molding having a tristar cross-section with rounded tips and with rounded corners, wherein the tristar cross-section has a geometric center C, a tip basis b, a tip height i, a height h, and a chord length k, and wherein each of the tips of the tristar cross-section is rounded by an arc of a circle, and wherein each of the corners of the tristar cross-section is rounded by an arc of a circle.

FIG. 7: shows a schematic of a molding having a tristar cross-section with rounded tips and with rounded corners, wherein the molding has a height H.

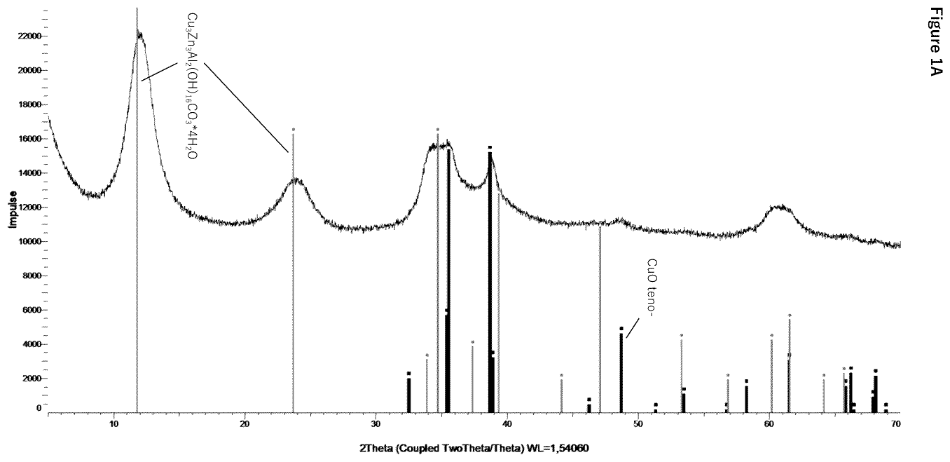

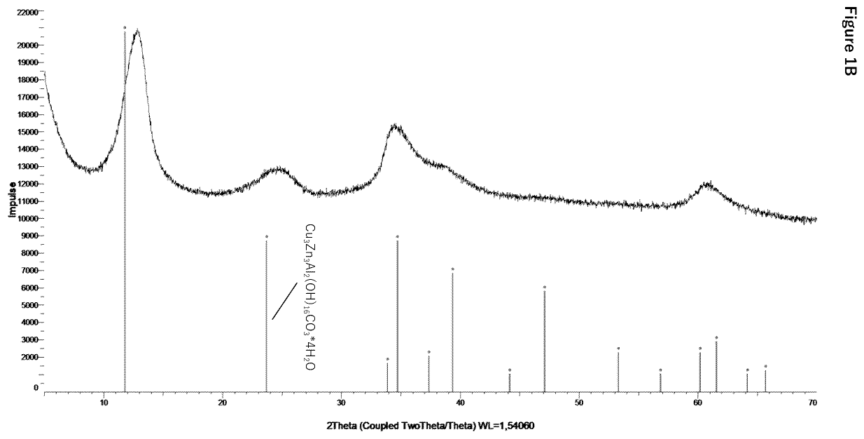

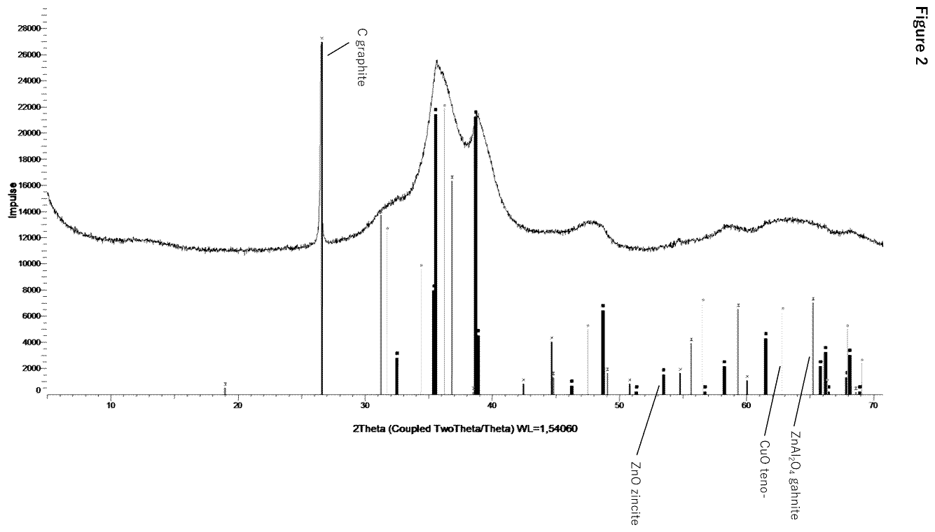

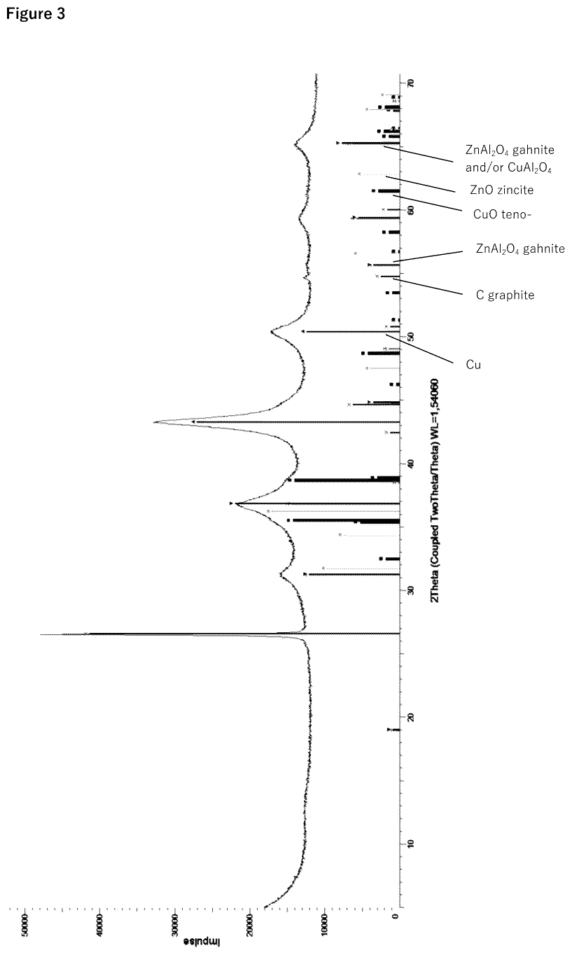

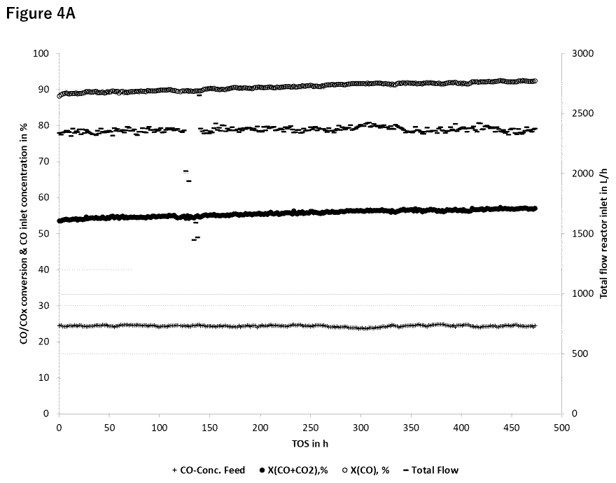

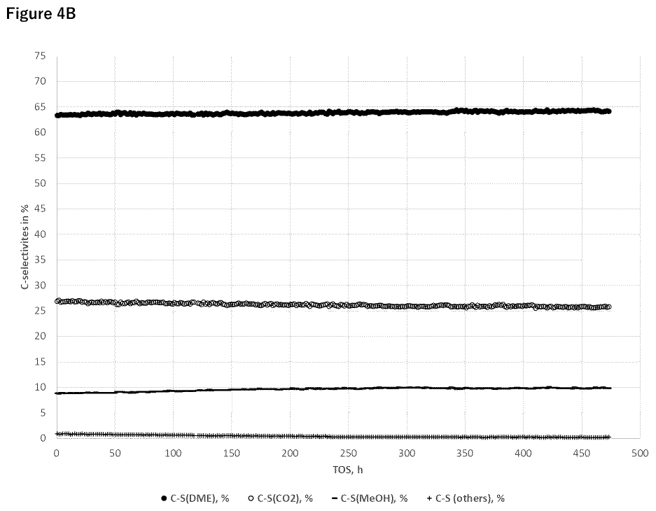

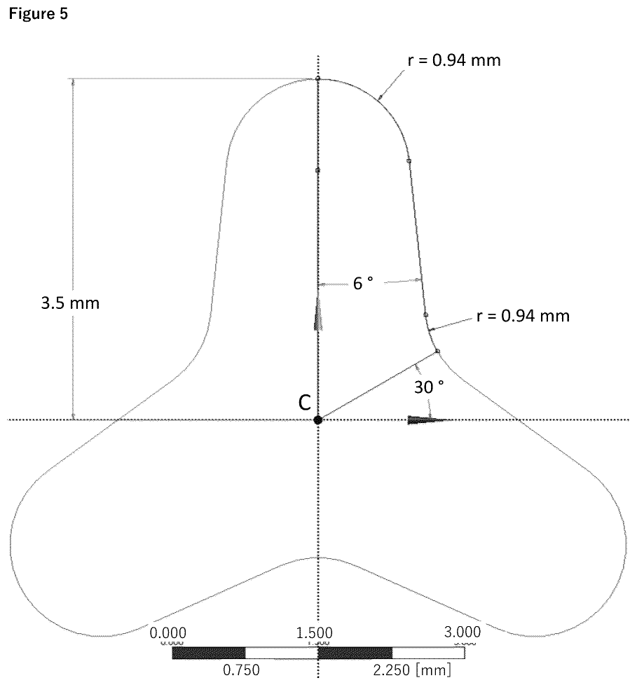

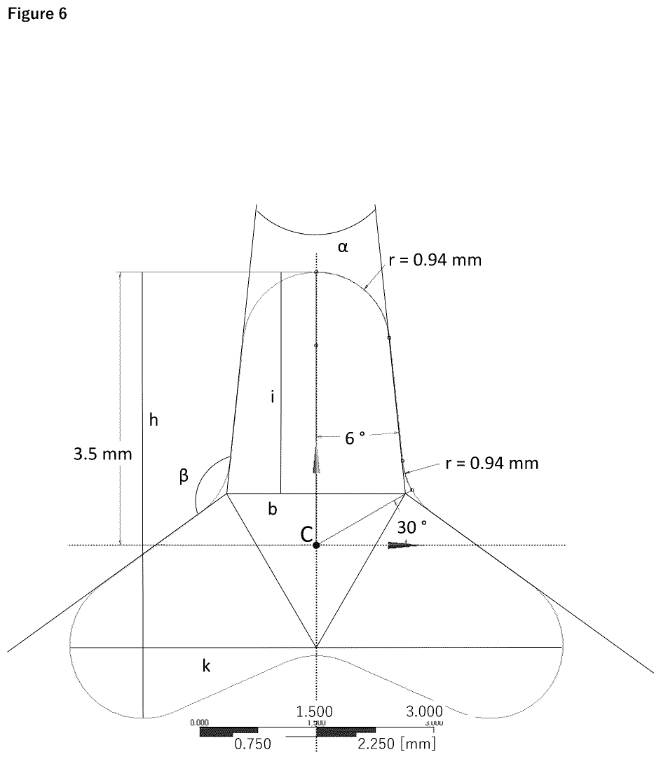

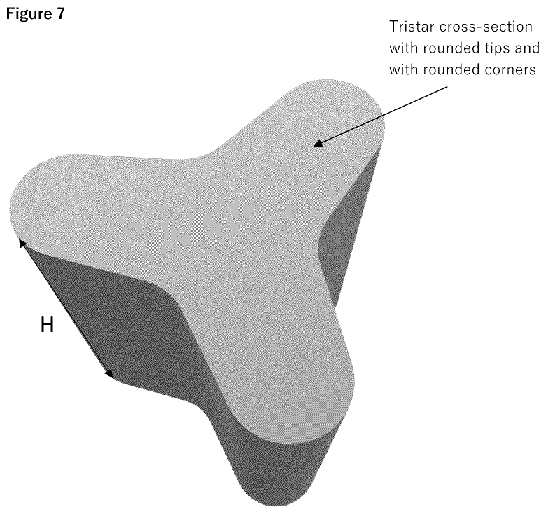

### CITED PRIOR ART

Wu et al. Catalysis Today 1998, vol. 45, p. 215-220
U.S. Pat. No. 6,048,820
EP 2857095 A1
WO 2020/212681 A1
EP 3727681 A1

1.-15. (canceled)

16. A supported copper catalyst comprising Cu, Zn, Al, Zr, Si, and O, wherein the catalyst comprises elemental copper, and wherein the catalyst displays a Zn:Si atomic ratio in the range of from 5:1 to 27:1,
the catalyst obtained by a process comprising the steps of:
(1) preparing a catalyst precursor comprising Cu, Zn, Al, Zr, Si, and O, according to a process comprising the steps of:
(i) preparing a first aqueous solution Si comprising one or more copper containing compounds, one or more zinc containing compounds, one or more aluminum containing compounds, and one or more zirconium containing compounds, wherein the pH of Si is in the range of from 0 to 5;
(ii) preparing a second aqueous solution S2, wherein the pH of S2 is adjusted to a value in the range of from 8.5 to 12;
(iii) preparing an aqueous mixture M comprising a solid suspended in water, comprising feeding Si into S2 under agitation, wherein during said feeding, the pH of the aqueous mixture M resulting from said feeding is kept in the range of from 8 to 10;
(iv) aging the aqueous mixture M obtained from (iii) for a duration in the range of from 1 to 12 h; and
(v) separating the solid from the aqueous mixture obtained from (iv);
wherein one or more silicon containing compounds are added for preparing S2 according to (ii), or wherein one or more silicon containing compounds are added to M during the aging of M according to (iv), or wherein one or more silicon containing compounds are added for preparing S2 according to (ii) and one or more silicon containing compounds are added to the mixture M during aging of M according to (iv);
(2) reducing the catalyst precursor obtained from (1) in an atmosphere comprising hydrogen for obtaining the supported copper catalyst.

17. The supported copper catalyst of claim 16, wherein the supported copper catalyst comprises one or more oxides of Zn, Al, Zr, and Si.

18. The supported copper catalyst of claim 16, wherein the supported copper catalyst displays a Cu:Zn:Al:Zr:Si molar ratio in the ranges of (50-59):(15-21):(22-30):(1-3):(0.8-4).

19. A catalyst precursor comprising Cu, Zn, Al, Zr, Si, and O, wherein the catalyst precursor displays a Zn:Si atomic ratio in the range of from 5:1 to 27:1,
the catalyst precursor obtained by a process comprising the steps of:
(i) preparing a first aqueous solution S1 comprising one or more copper containing compounds, one or more zinc containing compounds, one or more aluminum containing compounds, and one or more zirconium containing compounds, wherein the pH of S1 is in the range of from 0 to 5;
(ii) preparing a second aqueous solution S2, wherein the pH of S2 is adjusted to a value in the range of from 8.5 to 12;
(iii) preparing an aqueous mixture M comprising a solid suspended in water, comprising feeding S1 into S2 under agitation, wherein during said feeding, the pH of the aqueous mixture M resulting from said feeding is kept in the range of from 8 to 10;
(iv) aging the aqueous mixture M obtained from (iii) for a duration in the range of from 1 to 12 h; and
(iv) separating the solid from the aqueous mixture obtained from (iv);
wherein one or more silicon containing compounds are added for preparing S2 according to (ii), or wherein one or more silicon containing compounds are added to M during the aging of M according to (iv), or wherein one or more silicon containing compounds are added for preparing S2 according to (ii) and one or more silicon containing compounds are added to the mixture M during aging of M according to (iv).

20. The catalyst precursor of claim 19, wherein the catalyst precursor comprises one or more hydroxycarbonate mixed oxides comprising two or more of Cu, Zn, and Al; or wherein the catalyst precursor comprises one or more oxides of Cu, Zn, Al, Zr, and Si.

21. The catalyst precursor of claim 19, wherein the catalyst precursor comprises Zr-modified Cu₃Zn₃Al₂(OH)₁₆CO₃, wherein the 003 reflection in the x-ray diffractogram of the Zr-modified Cu₃Zn₃Al₂(OH)₁₆CO₃ is shifted to higher ° 2Theta values compared to the x-ray diffractogram of Cu₃Zn₃Al₂(OH)₁₆CO₃, wherein the 003 reflection in the x-ray diffractogram of the Zr-modified Cu₃Zn₃Al₂(OH)₁₆CO₃ is located in the range of from 11 to 13.5° 2Theta.

22. The catalyst precursor of claim 19, wherein the catalyst precursor displays a Cu:Zn:Al:Zr:Si molar ratio in the ranges of (50-59):(15-21):(22-30):(1-3):(0.8-4).

23. A process for the preparation of a catalyst precursor comprising Cu, Zn, Al, Zr, Si, and O, the process comprising:
(i) preparing a first aqueous solution Si comprising one or more copper containing compounds, one or more zinc containing compounds, one or more aluminum containing compounds, and one or more zirconium containing compounds, wherein the pH of Si is in the range of from 0 to 5;
(ii) preparing a second aqueous solution S2, wherein the pH of S2 is adjusted to a value in the range of from 8.5 to 12;
(iii) preparing an aqueous mixture M comprising a solid suspended in water, comprising feeding Si into S2 under agitation, wherein during said feeding, the pH of the aqueous mixture M resulting from said feeding is kept in the range of from 8 to 10;
(iv) aging the aqueous mixture M obtained from (iii) for a duration in the range of from 1 to 12 h; and
(v) separating the solid from the aqueous mixture obtained from (iv);
wherein one or more silicon containing compounds are added for preparing S2 according to (ii), or wherein one or more silicon containing compounds are added to M during the aging of M according to (iv), or wherein one or more silicon containing compounds are added for preparing S2 according to (ii) and one or more silicon containing compounds are added to the mixture M during aging of M according to (iv).

24. The process of claim 23, wherein the one or more silicon containing compounds comprise sodium water glass.

25. The process of claim 23, wherein the process further comprises:
(vi) washing the solid obtained from (v); and/or
(vii) drying the solid obtained from (v) or (vi); and/or
(viii) calcining the solid obtained from (v), (vi), or (vii).

26. The process of claim 23, wherein in (iii) the pH of the aqueous mixture M is kept in the pH range by metering a third aqueous solution S3 into the aqueous mixture M, wherein the pH of S3 is in the range of from 11 to 14.

27. A process for the preparation of a supported copper catalyst comprising Cu, Zn, Al, Zr, Si, and O, wherein the catalyst comprises elemental copper, the process comprising:
(1) preparing a catalyst precursor comprising Cu, Zn, Al, Zr, Si, and O, according to the process of claim 25;
(2) reducing the catalyst precursor obtained from (1) in an atmosphere comprising hydrogen for obtaining the supported copper catalyst.

28. A method for the conversion of CO₂-containing syngas to methanol, the method comprising:
(A) providing a supported copper catalyst according to claim 16;
(B) preparing a gas mixture comprising CO, H₂, and CO₂;
(C) contacting the supported copper catalyst provided in (A) with the gas mixture prepared in (B), wherein the contacting is performed at a temperature in the range of from 200 to 350° C.

29. A method for the conversion of syngas to dimethyl ether, the method comprising:
(A) mixing a supported copper catalyst according to claim 16 with an acidic co-catalyst;
(B) preparing a gas mixture comprising CO, H₂, and CO₂;
(C) contacting the catalyst mixture prepared in (A) with the gas mixture prepared in (B), wherein the contacting is performed at a temperature in the range of from 200 to 300° C.

30. A method for utilizing a supported copper catalyst according to claim 16 as a reverse water-gas shift catalyst, in the reforming of methanol, in the conversion of CO₂-containing syngas to methanol, in the reforming of dimethyl ether, and in the conversion of syngas to dimethyl ether.
